(19) United States
(12) Patent Application Publication
(10) Pub. No.: US 2026/0320783 A1
(43) Pub. Date: Oct. 15, 2026

(54) METHOD FOR TRAINING A MODEL CAPABLE OF ANALYZING HIGH-RESOLUTION IMAGE BASED ON HIERARCHICAL PATCH SELECTION, METHOD AND SYSTEM FOR PROVIDING DIAGNOSIS RESPONSES BASED ON HIGH-RESOLUTION IMAGE ANALYSIS

(71) Applicant: LG MANAGEMENT DEVELOPMENT INSTITUTE CO., LTD., Seoul (KR)

(72) Inventors: Jong Seong JANG, Gyeonggi-do (KR); Bum Soo KIM, Seoul (KR); Soon Young LEE, Seoul (KR); Jun Hyun LEE, Seoul (KR)

(21) Appl. No.: 19/466,146

(22) Filed: Jan. 30, 2026

Related U.S. Application Data
(63) Continuation of application No. PCT/KR2025/013318, filed on Aug. 29, 2025.

(30) Foreign Application Priority Data
Apr. 29, 2024 (KR) 10-2024-0116881
Aug. 28, 2025 (KR) 10-2025-0121360
Aug. 28, 2025 (KR) 10-2025-0121528

(51) Int. Cl. G06T 7/00; G06N 3/042; G06N 3/08

(52) U.S. Cl. CPC G06T 7/0012; G06N 3/042; G06N 3/08; G06T 2207/20081; G06T 2207/20084; G06T 2207/30024

(57) ABSTRACT

A method and system for providing a diagnosis response based on high-resolution image analysis may perform operations comprising: receiving an activation signal for accessing data stored in memory; in response to the receiving of the activation signal, accessing a diagnostic image stored in the memory; loading the diagnostic image from the memory; generating at least one response to the diagnostic image using at least one artificial intelligence model configured to receive the diagnostic image as an input, the at least one artificial intelligence model being pre-trained to perform image analysis based on data that integrates one or more of low-magnification features and one or more of high-magnification features of the diagnostic image; and inputting the at least one response to the diagnostic image to at least one subsequent processing component.

### CROSS-REFERENCE TO RELATED APPLICATIONS

This application is a continuation of International Patent Application No. PCT/KR2025/013318, filed on Aug. 29, 2025, which claims the benefit of and priority to Korean Patent Application No. 10-2024-0116881, filed on Aug. 29, 2024, and Korean Patent Application No. 10-2025-0121360, filed on Aug. 28, 2025, the entire disclosures of which are hereby incorporated herein by reference in their entireties.

### BACKGROUND

### Technical Field

The present disclosure generally relates to a method for training a high-resolution image analysis model based on hierarchical patch selection, and a method and system for providing a diagnosis response based on high-resolution image analysis. More specifically, some embodiments of the present disclosure relate to a method for training a high-resolution image analysis model based on hierarchical patch selection, and a method and system for providing a diagnosis response based on high-resolution image analysis capable of alleviating the dilemma of difficulty in capturing clues necessary for diagnosis from low-magnification images of high-resolution images and excessive cost in analyzing high-magnification images.

The present disclosure also generally relates to a method for training a time-series analysis model for a plurality of high-resolution images, and a method and system for providing a diagnosis response based on time-series analysis for the plurality of high-resolution images. More specifically, certain embodiments of the present disclosure relate to a method for training a time-series analysis model for a plurality of high-resolution images, and a method and system for providing a diagnosis response based on time-series analysis for the plurality of high-resolution images capable of capturing change patterns in a plurality of high-resolution images through time-series analysis of the plurality of high-resolution images photographed at a plurality of points in time, and providing a predictive diagnosis response to the plurality of high-resolution images based thereon.

### Related Art

Understanding and interpreting high-resolution images are an important process in various fields requiring problem diagnosis and analysis. However, due to their complexity and vast amount of information, the high-resolution images require skilled analysis of an expert and massive computational resources. The diagnostic and analysis standards, which rely on expert knowledge, are subject to continuous change with the emergence of new technologies and the change in requirements.

Recent advances in digitalization have led to the introduction of various computational methodologies to automate image analysis. In particular, deep learning has proven effective in understanding complex and atypical images. However, existing deep learning models tend to operate independently without interaction between an expert and an algorithm, due to lack of interpretability and limitations in the ability to infer from the derived results.

This issue is particularly prominent in the analysis of tissue pathology images. Understanding and interpreting the tissue pathology images are an important process in cancer diagnosis. However, due to the complexity of the tissue pathology images, image examination requires skill and extensive resources. Furthermore, pathologists need long-term training to fully understand a medical context.

To address this issue, a method of segmenting a whole slide image (WSI) of a tissue pathology image into a plurality of non-overlapping patches, extracting features of each patch using a pre-trained feature extractor such as Residual Network (ResNet) or Vision Transformer, and then using an aggregator model to synthesize the extracted features to ultimately perform a slide level prediction has been developed.

However, this method implies limitations of ineffectively utilizing the multi-resolution characteristics of the WSI. Pathologists grasp the overall tissue structure (tissue architecture) of a tissue pathology image at low magnification and then inspect detailed cellular features (cellular structures) at high magnification to make diagnostic determination. Existing models may fail to effectively mimic this hierarchical analysis, miss high-frequency components that serve as diagnostic clues at low magnifications, and face the multi-resolution dilemma, when the number of patches increases exponentially at high magnification, thereby increasing analysis cost.

To address this issue, several multi-resolution analysis techniques have been proposed. Methods such as a method of modeling the WSI as a hierarchical graph to analyze the correlation between patches and using low-magnification features to select important patches at high magnifications have been known. However, these methods incur excessive computational complexity or dilute high-frequency components that serve as important clues in low-magnification images, resulting in reduced accuracy of patch selection.

Accordingly, there is a growing need for new technologies capable of overcoming the limitations of existing models, such as lack of interpretability and limitations in the ability to infer, and the multi-resolution dilemma, and bridging the gap between expert knowledge and computational models to enhance the efficiency and reliability of image analysis.

In order to comprehensively grasp the changes in the state of an image over time, it is necessary to analyze a plurality of images photographed at a plurality of points in time in a time series. While the conventional method of segmenting a high-resolution image into a plurality of patches and integrating the features of each patch to ultimately perform an overall image-level prediction may perform well on individual images, there may be limitations in accurately capturing change patterns of the plurality of images.

Time-series analysis is a technique suited for analyzing data that occurs in temporal order. Traditionally, models such as recurrent neural networks (RNNs) or long short-term memory (LSTMs) have been used to learn patterns and long-term dependencies in time-series data. However, these models have limitations such as computational inefficiency or dilution of important past information as the length of the input sequence increases.

Recently, attention-based models such as Transformers have been introduced to time-series data analysis to effectively address these issues. The transformers utilize a self-attention mechanism that learns relationships between all positions in the input sequence in parallel, thereby accurately capturing important temporal dependencies in long sequences. This technology offers new possibilities for more accurately predicting change trends in image by performing time-series analysis on a series of a plurality of high-resolution images.

### SUMMARY

According to various embodiments of the present disclosure, a method for training a high-resolution image analysis model, and a method and system for providing a diagnosis response based on high-resolution image analysis may effectively alleviate a multi-resolution dilemma and improve prediction performance and reliability by capturing diagnostic clues in low-magnification images through spectral analysis of high-resolution images and efficiently selecting patches in high-magnification images based thereon.

According to various embodiments of the present disclosure, a method for training a high-resolution image analysis model, and a method and system for providing a diagnosis response based on high-resolution image analysis may effectively encode geometric positional relationships and morphological similarities between patches by considering both positional information and frequency characteristic information of a plurality of multiple patches in computing patch relationship information used to efficiently select patches of high-magnification images, and improve the computational efficiency of high-magnification image analysis based thereon.

According to various embodiments of the present disclosure, a method for training a high-resolution image analysis model, and a method and system for providing a diagnosis response based on high-resolution image analysis may effectively extend an existing single-resolution analysis system to multi-resolution and improve prediction performance and reliability by using a cross-attention mechanism in a process of integrating features of multi-magnification images and explicitly modeling causal relationship between multi-magnification by introducing unexplained factors in a latent space down-sampling process as noise terms.

According to various embodiments of the present disclosure, a method for training a time-series analysis model for a plurality of high-resolution images, and a method and system for providing a diagnosis response based on time-series analysis for the plurality of high-resolution images may efficiently analyze high-resolution images at each point in time when the plurality of high-resolution images were photographed based on hierarchical patch selection, organizing a plurality of analysis results for the plurality of high-resolution images into time-series data, and training the same with the time-series analysis model, thereby providing a precise predictive diagnosis response that reflects temporal changes.

However, the purposes or objects of the present disclosure are not limited to those as described above.

An embodiment provides a computer-implemented method comprising: receiving an activation signal for accessing data in at least one memory; accessing a data structure in the memory in response to the reception of the activation signal, the data structure comprising a diagnostic image; loading the diagnostic image from the memory; generating, by at least one processor, at least one response to the diagnostic image using at least one artificial intelligence model that takes the diagnostic image as an input, the at least one artificial intelligence model being pre-trained to perform image analysis based on data that integrates some of low-magnification features and some of high-magnification features of the diagnostic image; and inputting the at least one response to at least one subsequent processing component.

In another aspect, the method may further include manifesting, by the at least one subsequent processing component, the at least one response through at least one user interface.

In another aspect, the method may further include providing, through the user interface, an image in which a partial area of the diagnostic image is highlighted, the partial area of the diagnostic image corresponding to some of the low-magnification features and some of the high-magnification features of the diagnostic image utilized in an arithmetic operation for determining the response.

In another aspect, the response may have either one form of interactive text, voice, or a combination thereof.

In another aspect, the method may further include: receiving a plurality of diagnostic images according to a user input; receiving a plurality of pieces of point-in-time information for the plurality of diagnostic images; inputting the plurality of diagnostic images and the plurality of pieces of point-in-time information to at least one pre-trained artificial intelligence model; and providing a user with a response output for the plurality of diagnostic images output from the at least one artificial intelligence model.

In another aspect, each of the plurality of diagnostic images may include a tissue pathology image for the user, and the response output may include at least one of a progression of a specific disease of the user, a grade of the disease, a likelihood of recurrence of the disease, or a future change trend of the disease.

In another aspect, the method for pre-training the at least one artificial intelligence model for the plurality of diagnostic images may include: outputting a plurality of analysis prediction values for the plurality of diagnostic images by performing the image analysis on each of the plurality of diagnostic images photographed at a plurality of points in time using the at least one artificial intelligence model; configuring the plurality of analysis prediction values into time-series data; and training the at least one artificial intelligence model for the plurality of diagnostic images to output predictive diagnosis responses for the plurality of diagnostic images using the time-series data based on the plurality of analysis prediction values as an input.

In another aspect, the configuring the plurality of analysis prediction values into the time-series data may include: aligning the plurality of analysis prediction values according to an order of the plurality of points in time; generating a positional embedding, which includes time information of a point in time when the corresponding diagnostic image has been photographed, for each of the aligned plurality of analysis prediction values; and inputting the aligned plurality of analysis prediction values, which includes the positional embedding, to the at least one artificial intelligence model for the plurality of diagnostic images.

In another aspect, the method for pre-training the at least one artificial intelligence model may include: generating a plurality of first patch feature embeddings including low-magnification features and a plurality of second patch feature embeddings including high-magnification features from the diagnostic image; generating a plurality of patch relationship information embeddings including relationship information between a plurality of patches from the plurality of patches of the diagnostic image; selecting some of the plurality of first patch feature embeddings based on the plurality of patch relationship information embeddings; selecting some of the plurality of second patch feature embeddings based on the plurality of patch relationship information embeddings; and training the at least one artificial intelligence model comprising a prediction module so that a difference between a prediction value output by the prediction module based on the selected some of the plurality of first patch feature embeddings and some of the plurality of second patch feature embeddings and ground truth data is minimized.

In another aspect, the generating the plurality of patch relationship information embeddings may include: generating a distance-based adjacency matrix (A_{dis}) based on distance information for the plurality of patches of a low-magnification image for the diagnostic image; generating a frequency feature-based adjacency matrix (A_{HF}) based on similarity information between frequency features for the plurality of patches of the low-magnification image for the diagnostic image; and generating the plurality of patch relationship information embeddings including positional information and characteristic relationship information between the plurality of patches by utilizing the distance-based adjacency matrix and the frequency feature-based adjacency matrix.

In another aspect, the generating the frequency feature-based adjacency matrix (A_{HF}) may include: computing similarity between a plurality of frequency feature embeddings for the plurality of patches of the low-magnification image; and generating the frequency feature-based adjacency matrix (A_{HF}) based on the similarity information between a plurality of frequency embeddings and the distance information between the plurality of patches of the low-magnification image.

In another aspect, the generating the plurality of patch relationship information embeddings may include: acquiring a plurality of positional feature vectors encoding geometric positional information between the plurality of patches of the low-magnification image by performing spectral analysis on the distance-based adjacency matrix; and generating the plurality of patch relationship information embeddings by performing a neural network arithmetic operation that learns a graph structure on some of the acquired plurality of positional feature vectors selected according to a predetermined condition and the frequency feature-based adjacency matrix.

In another aspect, the acquiring the plurality of positional feature vectors may include computing a plurality of eigenvectors for a graph Laplacian of the distance-based adjacency matrix as the plurality of positional feature vectors according to the following equation:

$$Δ = {I}_{N} - {D}_{dis}^{- 1 / 2} {A}_{dis} {D}_{dis}^{- 1 / 2} = {U}^{T} Λ U \qquad ( Equation )$$

(wherein, A is the graph Laplacian, Iₙ is an identity matrix, D_{dis} is a degree matrix, A_{dis} is the distance-based adjacency matrix, U is an eigenvector matrix, and A is an eigenvalue matrix.)

In another aspect, the generating the plurality of first patch feature embeddings and the plurality of second patch feature embeddings may include: generating the plurality of first patch feature embeddings by integrating a plurality of first visual feature embeddings and a plurality of first frequency feature embeddings for the low-magnification image of the diagnostic image; and generating the plurality of second patch feature embeddings by integrating a plurality of second visual feature embeddings and a plurality of second frequency feature embeddings for the high-magnification image of the diagnostic image.

In another aspect, the selecting some of the plurality of first patch feature embeddings may include: computing a plurality of weights for the plurality of patches of the low-magnification image based on a plurality of patch integrated embeddings that integrate the plurality of first visual feature embeddings, the plurality of first frequency feature embeddings, and the plurality of patch relationship information embeddings; and selecting k first patch feature embeddings corresponding to the top k weights (k is a positive integer) among the plurality of weights, from the plurality of first patch feature embeddings.

In another aspect, the selecting some of the plurality of second patch feature embeddings may include: computing a plurality of weights for the plurality of patches of the high-magnification image based on a plurality of patch integrated embeddings that integrate the plurality of second visual feature embeddings, the plurality of second frequency feature embeddings, and the plurality of patch relationship information embeddings; and selecting k×M² second patch feature embeddings corresponding to the top k×M² weights (M is a relative magnification of a high magnification to a low magnification) among the plurality of weights, from the plurality of second patch feature embeddings.

In another aspect, the training the at least one artificial intelligence model including the prediction module may include: computing a first weighted sum of the selected k first patch feature embeddings based on the top k weights for the plurality of patches of the low-magnification image; computing a second weighted sum of the selected k×M² second patch feature embeddings based on the top k×M² weights for the plurality of patches of the high-magnification image; and training the at least one artificial intelligence model including the prediction module such that the difference between the prediction value output by the prediction module by using a predetermined arithmetic operation result for the first weighted sum and the second weighted sum and the ground truth data is minimized.

An embodiment provides a system including: at least one memory; and at least one processor for executing a method for providing a response to a diagnostic image by reading at least one instruction stored in the at least one memory, wherein the at least one instruction includes instructions for: receiving an activation signal for accessing at least one piece of data in the memory; accessing a data structure in the memory in response to receiving the activation signal, wherein the data structure includes a diagnostic image; loading the diagnostic image from the memory; generating, by at least one processor, at least one response to the diagnostic image using at least one artificial intelligence model that takes the diagnostic image as an input, wherein the at least one artificial intelligence model is pre-trained to perform image analysis based on data that integrates some of low-magnification features and some of high-magnification features of the diagnostic image; and inputting the at least one response to at least one subsequent processing component.

In another aspect, the system may further include: a plurality of neurons configured as an array including at least one register, at least one programmable logic, and at least one input interface; a plurality of synaptic circuits storing synaptic weights that adjust concatenation strengths between the plurality of neurons; and at least one routing network that controls data flow between the plurality of neurons, wherein each of the plurality of neurons may be concatenated to at least another neuron through the routing network and may further include a field programmable gate array (FPGA) implementation for a predetermined artificial neural network that establishes a transfer path for the weights.

In another aspect, the system may further include: a plurality of neurons organized as an array including at least one register, at least one microprocessor, and at least one input; and a plurality of synaptic circuits storing synaptic weights that adjust concatenation strengths between the plurality of neurons, wherein each of the plurality of neurons may further include an application specific integrated circuit (ASIC) for a predetermined artificial neural network concatenated to at least another neuron through any one of the plurality of synaptic circuits.

According to various embodiments of the present disclosure, based on time-series analysis of a plurality of high-resolution images, image change patterns can be predicted, thereby increasing the reliability of predictive diagnosis responses. While existing models only provide static predictions for a single image, a method for training a time-series analysis model for the plurality of high-resolution images, a method and system for providing a time-series analysis-based diagnosis response for the plurality of high-resolution images according to various embodiments of the present disclosure may configure and learn a plurality of analysis results for a plurality of points in time of the plurality of high-resolution images into time-series data, thereby comprehensively grasping temporal change patterns and more accurately predicting prediction values corresponding to change patterns of the images.

According to various embodiments of the present disclosure, for a plurality of high-resolution images photographed at different points in time, position encodings containing time information at which each image was acquired are generated. The position encodings containing the time information is then input to a time-series analysis model along with the analysis prediction values for each of the plurality of high-resolution images, enabling the model to efficiently learn the temporal dependencies (long-term dependency) of the plurality of high-resolution images. Accordingly, this may enable the time-series analysis model to accurately capture important temporal dependencies even as the length of input sequence increases.

According to various embodiments of the present disclosure, by efficiently analyzing each of the plurality of high-resolution images photographed at a plurality of points in time based on hierarchical patch selection and configurating the results into time-series data, the computational complexity required to analyze all patches can be reduced while still obtaining precise prediction results. This may improve or maximize the accuracy of time-series analysis of the plurality of high-resolution images.

According to various embodiments of the present disclosure, diagnostic clues in low-magnification images can be captured through spectral analysis of a plurality of patches in low-magnification images. Based thereon, the multi-resolution dilemma can be effectively alleviated by efficiently selecting some of the plurality of patches in a high-magnification image that have a large influence on the prediction results.

According to various embodiments of the present disclosure, by analyzing the plurality of patches of low-magnification images at relatively low magnification compared to high-resolution images, clues important for diagnosis can be captured from low-magnification images. Based thereon, Top-K patches can be selected to focus only on important areas instead of exploring all high-magnification patches. This may reduce graphics processing unit (GPU) memory usage and inference time, and improve model performance and computational efficiency.

According to various embodiments of the present disclosure, a method for training a high-resolution image analysis model can explicitly learn causal relationships between multiple magnifications by using a cross-attention mechanism based on a plurality of patch feature embeddings from low-magnification images and a plurality of patch feature embeddings from high-magnification images, and introducing unexplained factors from a down-sampling process from high to low magnification as noise terms in a latent space. This effectively extends existing single-resolution analysis systems to multi-resolutions and contributes to improved prediction performance and reliability.

According to various embodiments of the present disclosure, the interpretability and reliability of the decision-making process of a high-resolution image analysis model can be enhanced. By generating patch relationship information, including positional and feature relationship information between the plurality of patches, the positional and morphological similarities between patches are structurally encoded. The process of selecting important patches using a gated attention mechanism based on patch relationship information can provide information that experts can easily understand on what basis the model reaches its conclusions. This facilitates interaction between the model and the expert and enhances the reliability of image analysis.

However, the technical effect of the present disclosure are not limited to those mentioned above, and other benefits not mentioned may be clearly understood from the following description.

### BRIEF DESCRIPTION OF DRAWINGS

FIG. 1 illustrates a block diagram of a computing system implementing a high-resolution image analysis-based diagnosis response provision service according to an embodiment.

FIG. 2 is a schematic diagram illustrating the structure of a neuromorphic circuit that is included in a processor according to an embodiment.

FIG. 3 is a block diagram illustrating a computing device implementing a high-resolution image analysis-based diagnosis response provision service according to an embodiment.

FIG. 4 is a block diagram illustrating a computing device implementing a high-resolution image analysis-based diagnosis response provision service according to an embodiment.

FIG. 5 is a block diagram illustrating a computing device implementing a high-resolution image analysis-based diagnosis response provision service according to an embodiment.

FIG. 6 is a conceptual diagram for illustrating the generation of a predictive diagnosis response by a time-series analysis-based predictive diagnosis response generation module according to an embodiment.

FIG. 7 is a block diagram illustrating an exemplary configuration of a hierarchical patch selection-based image analysis module utilized to implement a high-resolution image analysis-based diagnosis response provision service according to an embodiment.

FIG. 8 illustrates an exemplary configuration of a spectral analysis-based multi-resolution analysis architecture for a high-resolution image according to an embodiment.

FIG. 9 is a conceptual diagram for illustrating a method for extracting positional information and characteristic relationship information between a plurality of patches of a high-resolution image according to an embodiment.

FIG. 10 is a conceptual diagram illustrating a framework for a multi-resolution integrated model that reflects causal relationships between multiple magnifications according to an embodiment.

FIG. 11 is a configuration diagram illustrating a framework for a multi-resolution integrated model that reflects causal relationships between multiple magnifications according to an embodiment.

FIG. 12 is a flowchart of a method for training a high-resolution image analysis model according to an embodiment.

FIG. 13 is a flowchart for illustrating a step of generating a plurality of patch relationship information embeddings in FIG. 12 according to an embodiment.

FIG. 14 is a flowchart of a method for providing a diagnosis response based on high-resolution image analysis according to an embodiment.

FIG. 15 is a flowchart of a method for training a time-series analysis model for a plurality of high-resolution images according to an embodiment.

FIG. 16 is a flowchart of a step of configuring a plurality of analysis prediction values into time-series data in FIG. 15 according to an embodiment.

FIG. 17 is a flowchart of a method for providing a diagnosis response based on time-series analysis for a plurality of high-resolution images according to an embodiment.

### DETAILED DESCRIPTION OF EMBODIMENTS

Various embodiments of the present disclosure may impose various transformations, and specific embodiments illustrated in the drawings will be described in detail in the detailed description. The advantages, features and methods for achieving the same will become apparent from the following description of the embodiments given in conjunction with the accompanying drawings. However, various embodiments of the present disclosure are not limited to the embodiments described herein but may be embodied in many different forms. It will be understood that, although the terms “first” or “second” may be used herein to distinguish one component from another component, these components should not be limited by these terms. In addition, a singular expression includes a plural expression, unless the context clearly states otherwise. In addition, it should be understood that the terms such as “include” or “have” are merely intended to indicate that features, or components described in the specification are present, and are not intended to exclude the possibility that one or more other features, or components will be added. In addition, components in the drawings may be exaggerated or shrunk for the convenience of descriptions. For example, since the size and thickness of each element in the drawings has been arbitrarily modified for the convenience of descriptions, it should be noted that various embodiments of the present disclosure are not necessarily limited to what has been shown in the drawings.

Hereinafter, various embodiments of the present disclosure will be described in detail with reference to appended drawings. Throughout the specification, the same or corresponding component is assigned the same reference numeral, and repeated descriptions thereof will be omitted.

—System 1000 Providing High-Resolution Image Analysis-Based Diagnosis response Provision Service

A system 1000 according to an embodiment is configured to efficiently analyze the features of patches in a high-resolution image and perform diagnostic prediction. In particular, in order to alleviate the multi-resolution dilemma of a whole slide image (WSI), the system 1000 utilizes a hierarchical analysis method that utilizes features of both low- and high-magnification images.

The system 1000 receives a high-resolution image, segments the high-resolution image into low- and high-magnification patches, and extracts both original features and high-frequency features from each patch. The system 1000 then generates a learnable geometric position encoding (LGPE) containing information on the geometric positional relationships and morphological similarity between the patches. Based thereon, the system 1000 may efficiently select diagnostically significant Top-K patches from a low-magnification image.

Furthermore, the system 1000 may finally aggregate the features of the high-magnification patches corresponding to the selected low-magnification patches and output a prediction value for the high-resolution image. This system 1000 may provide a diagnosis response with high accuracy and reliability while reducing the computational complexity required to analyze all high-magnification patches through this process.

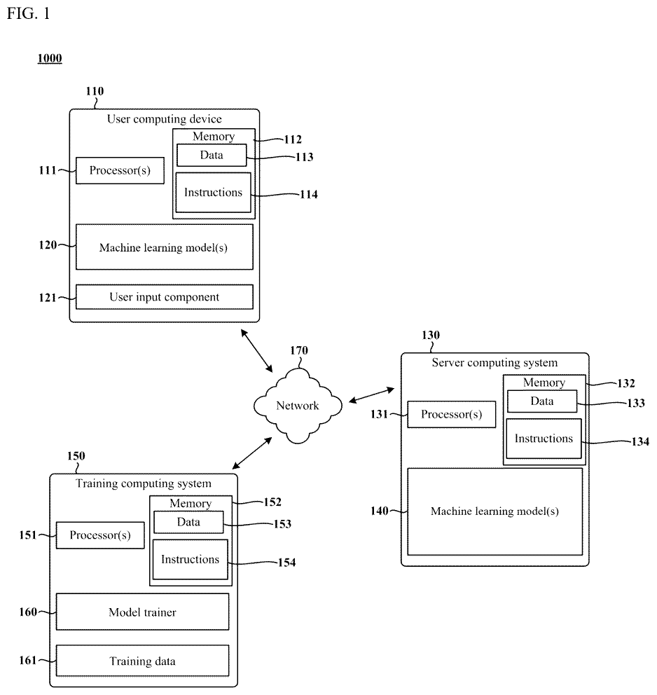

FIG. 1 illustrates a block diagram of a computing system implementing a high-resolution image analysis-based diagnosis response provision service according to an embodiment.

Referring to FIG. 1, the computing system or computer system 1000 which implements the high-resolution image analysis-based diagnosis response provision service according to an embodiment includes a user computing device or user computer 110, a server computing system or server computer 130, and a training computing system or training computer 150, and any devices which may configured to communicate through a network 170.

A method for providing a diagnosis response based on high-resolution image analysis according to an embodiment may (1) be implemented and provided locally by the user computing device 110, (2) implemented and provided in the form of a web or cloud service by the server computing system 130 which communicates with the user computing device 110, and (3) implemented and provided by mutual association of the user computing device 110 and the server computing system 130.

In an embodiment, the user computing device 110 and/or the server computing system 130 may train a machine learning model 120 and/or 140 through interaction with the training computing system 150 communicationally connected through the network 170. The training computing system 150 may be a system separated from the server computing system 130 or may be included in the server computing system 130.

In addition, the artificial intelligence model may be (1) directly trained locally by the user computing device 110, (2) trained while the server computing system 130 and the user computing device 110 interact with each other through the network 170, and (3) trained by using various training techniques and learning techniques by the separate training computing system 150. In addition, the method may also be implemented by a method in which the artificial intelligence model trained by the training computing system 150 is transmitted to the user computing device 110 and/or the server computing system 130 through the network 170, and is provided and updated.

In some embodiments, the training computing system 150 may be comprised in the server computing system 130 or the user computing device 110.

### —User Computing Device 110

The user computing device 110 may include various types of computers or computing devices such as a smart phone, a cellular phone, a digital broadcasting device, personal digital assistants (PDA), a portable multimedia player (PMP), a desktop, a wearable device, an embedded computing device, a tablet personal computer (PC), an augmented reality (VR) device and/or a virtual reality (AR) device.

The user computing device 110 may include at least one processor 111 and memory 112. Herein, the processor 110 may be configured of one or a plurality of processors electrically connected. For example, the processor 110 may be a central processing unit (CPU), a graphics processing unit (GPU), application specific integrated circuits (ASICs), digital signal processors (DSPs), digital signal processing devices (DSPDs), programmable logic devices (PLDs), field programmable gate arrays (FPGAs), controllers, micro-controllers, microprocessors, and/or other electrical components.

In particular, depending on embodiments, the processor 111 may be configured based on an FPGA implementation, which is a hardware technology that implements a predetermined digital circuit, and/or the ASIC.

Herein, the FPGA implementation may refer to a flexible digital circuit programmable according to user needs.

In an embodiment, the FPGA implementation may include: a register that temporarily stores data and controls the flow and timing of signals to maintain arithmetic operation intermediate results or state information, thereby supporting synchronized operation of the FPGA; a programmable logic that programs an internal arithmetic operation of the FPGA to perform specific functions or arithmetic operations as configurable logic circuits according to user needs; and an input interface that receives signals from external devices or sensors and transfers the same to internal circuits as a path for accepting data from outside the FPGA.

By combining the above constituents, the FPGA implementation may provide flexible and diverse digital circuits.

The ASIC may refer to a customized integrated circuit fixed and designed to perform a specific purpose or function.

In an embodiment, the ASIC may include: a register that is a small memory device that temporarily stores and manages data and stores calculation intermediate results or state information to support to process an operation of the ASIC quickly; a microprocessor that is a central processing unit that performs control and arithmetic operations within the ASIC and performs multiple arithmetic operations or generates control signals to adjust an operation of the entire system as needed; and an input block that is an interface that receives data from the outside, receives data to be processed by the ASIC and transfers the same to the inside, and receives various pieces of input data through connections with sensors or external devices.

By combining the above constituents, the ASIC may perform specific-purpose tasks in an optimized manner.

For example, the ASICs may have the structure of a neuromorphic circuit in the form of an array containing a plurality of neuron circuits.

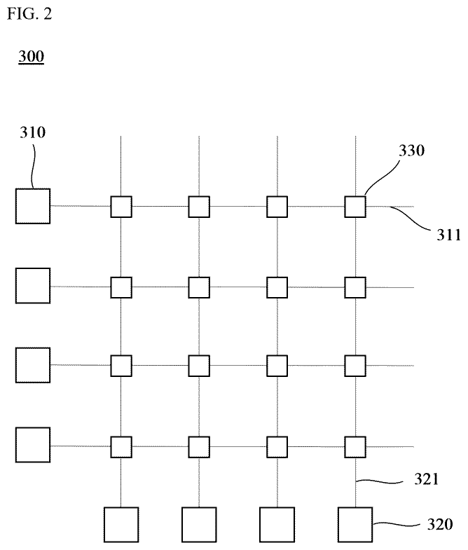

FIG. 2 is a schematic diagram illustrating the structure of a neuromorphic circuit 300 that may be included in a processor 111, 131, 151 according to an embodiment.

Referring to FIG. 2, the neuromorphic circuit 300 may include a plurality of presynaptic neuron circuits 310, a plurality of presynaptic lines 311 extending laterally from the plurality of presynaptic neuron circuits 310, a plurality of postsynaptic neuron circuits 320, a plurality of postsynaptic lines 321 extending longitudinally from the plurality of postsynaptic neuron circuits 320, and a plurality of synaptic circuits 330 provided at intersections of the plurality of presynaptic lines 311 and the plurality of postsynaptic lines 321.

The plurality of presynaptic neuron circuits 310 may transmit externally input signals in the form of electrical signals to the plurality of synaptic circuits 330 via the plurality of presynaptic lines 311.

The plurality of postsynaptic neuron circuits 320 may receive the electrical signals from the plurality of synaptic circuits 330 via the plurality of postsynaptic lines 321.

The plurality of postsynaptic neuron circuits 320 may transmit the electrical signals to the plurality of synaptic circuits 330 via the plurality of postsynaptic lines 321.

The plurality of synaptic circuits 330 may store weights included in layers configuring the neural network system implemented by the neuromorphic circuit 300 and perform predetermined arithmetic operations based on the weights and input data.

For example, each of the plurality of synaptic circuits 330 may include a resistive memory cell having a variable resistance. In this configuration, the resistance value of the plurality of synaptic circuits 330 may change according to the voltage applied through the plurality of presynaptic neuron circuits 310 or the plurality of post-synaptic neuron circuits 320, and weight data according to the resistance change may be stored.

The neuromorphic circuit 300 is configured or formed by simulating the structure of neurons and synapses, which are essential elements of the human brain. When a deep neural network (DNN) is realized using the neuromorphic circuit 300, the data processing speed may be improved and power consumption may be reduced compared to a case where the existing von Neumann structure is utilized.

The memory 112 may include one or more non-transitory and/or transitory computer-readable storage media, such as RAM, ROM, EEPROM, EPROM, flash memory devices, or magnetic disks, and combinations thereof, and may include web storage of servers performing storage functions of the storage or memory on the Internet. The memory 112 may store data 113 and instructions 114 necessary for the at least one processor 111 to perform a functional operation, such as training the artificial intelligence model or executing high-resolution image analysis through the artificial intelligence model.

In an embodiment, the user computing device 110 may store at least one machine learning model 120.

For example, in order to perform the method for providing the diagnosis response based on the high-resolution image analysis, the machine learning model 120 may be various machine learning models such as a plurality of neural networks (for example, the DNN) or other types of machine learning models, including non-linear models and/or linear models, and may be configured of a combination thereof.

For example, the machine learning models, such as linear regression, decision trees, random forests, gradient boosting, pre-trained language models, and/or deep learning models, may be stored. In addition, the neural network may include at least one of feed-forward neural networks, recurrent neural networks (for example, long short-term memory recurrent neural networks), convolutional neural networks and/or other forms of neural networks.

In addition, according to various embodiments, the user computing device 110 may store a model to be used in each process and a prompt template that serves as the basis for input to the model in order to perform at least a portion of the process performed for the method for providing the diagnosis response based on the high-resolution image analysis through a large language model (LLM).

In an embodiment, the user computing device 110 may receive at least one machine learning model 120 from the server computing system 130 via the network 170, store the machine learning model 120 in the memory 112, and then execute the stored machine learning model 120 by the processor 111 to perform the arithmetic operation for providing the diagnosis response based on the high-resolution image analysis.

In another embodiment, the server computing system 130 may include at least one machine learning model 140 and perform operations through the machine learning model 140, and may provide the high-resolution image analysis-based diagnosis response provision service to a user by linking with the user computing device 110 in a manner of communicating data related thereto with the user computing device 110.

For example, the user computing device 110 may perform the high-resolution image analysis-based diagnosis response provision service by providing an output for the input of a user using the machine learning model 140 through the server computing system 130 via the web.

In addition, the artificial intelligence model may also be implemented in such a way that at least some of the machine learning models 120 and/or 140 are executed on the user computing device 110 and the rest are executed on the server computing system 130.

In addition, the user computing device 110 may include at least one input component 121 that detects user input. For example, the user input component 121 may include a touch sensor (for example, a touch screen and/or a touch pad) that detects touch of an input medium of a user (for example, a finger or a stylus), an image sensor that detects a motion input of a user, a microphone, a button, a mouse and/or a keyboard that detects user voice input. In addition, the user input component 121 may include an interface and an external controller when receiving input from an external controller (for example, a mouse or a keyboard) through the interface.

### —Server Computing System 130

The server computing system 130 may perform a series of processes for providing the high-resolution image analysis-based diagnosis response provision service.

In an embodiment, the server computing system 130 may provide the high-resolution image analysis-based diagnosis response provision service by exchanging data necessary for a high-resolution image analysis-based diagnosis response provision service process to an external device, such as the user computing device 110, to drive on the external device.

More specifically, in an embodiment, the server computing system 130 may provide an environment in which an application for providing the high-resolution image analysis-based diagnosis response provision service may operate on the user computing device 110.

To this end, the server computing system 130 may include application programs, data, and/or instructions for the application to operate, and may transmit and receive various pieces of data based thereon to and from the external device.

The server computing system 130 may include at least one processor 131 and memory 132. Herein, the processor 131 may be configured of one or a plurality of processors electrically connected. For example, the processor 131 may comprise a central processing unit (CPU), a graphics processing unit (GPU), application specific integrated circuits (ASICs), digital signal processors (DSPs), digital signal processing devices (DSPDs), programmable logic devices (PLDs), field programmable gate arrays (FPGAs), controllers, micro-controllers, microprocessors, and/or other electrical components.

For example, the ASICs may have the structure of the neuromorphic circuit in the form of an array containing the plurality of neuron circuits (see FIG. 2).

In addition, the memory 132 may include one or more non-transitory and/or transitory computer-readable storage media, such as RAM, ROM, EEPROM, EPROM, flash memory devices, or magnetic disks, and combinations thereof. The memory 132 may store data 133 and instructions 134 required for the processors 131 to perform a functional operation such as the train of the artificial intelligence model or the execution of the method for providing the diagnosis response based on the high-resolution image analysis through the artificial intelligence model.

In an embodiment, the server computing system 130 may be implemented to include one or more computing devices or computers. For example, the server computing system 130 may be implemented so that a plurality of computing devices can operate according to sequential computing architecture, parallel computing architecture, or a combination thereof. Further, the server computing system 130 may include a plurality of computing devices connected through the network 170.

Further, the server computing device 130 may store one or more machine learning models 140. For example, the server computing system 130 may include a neural network and/or multilayer non-linear model as the machine learning model 140. An exemplary neural network may include a feed-forward neural network, a deep neural network, a recurrent neural network, and a convolution neural network.

In an embodiment, the server computing system 130 may further include a data store computing system (hereinafter, “data storage”), which is a storage for storing and managing raw data that serves as the basis for the high-resolution image analysis-based diagnosis response provision service.

The data storage may include various forms of data storage, ranging from file systems to cloud storage. For example, the data storage may include at least one database, for example, but not limited to, a relational database that uses a structured query language (SQL) to define and manipulate data; a NoSQL database that is designed for flexibility and scalability and processes unstructured and semi-structured data; a data warehouse that is a system used for reporting and data analysis, centralizes large amounts of data from multiple sources and is optimized for querying and analysis; a data warehouse that stores large amounts of raw data in basic formats such as structured data, semi-structured data, and unstructured data, and a local storage device or network attached storage (NAS) that stores data in files in a format generally accessible from a computer operating system.

### —Training Computing System 150

The training computing system 150 may include at least one processor 151 and memory 152. Herein, the processor 151 may be configured of at least one or a plurality of processors electrically connected. For example, the processor 151 may comprise the CPU, the GPU, the ASICs, the DSPs, the DSPDs, the PLDs, the FPGAs, controllers, micro-controllers, microprocessors, and/or other electrical components.

For example, the ASICs may have the structure of a neuromorphic circuit in the form of an array containing the plurality of neuron circuits (see FIG. 2).

In addition, the memory 152 may include one or more non-transitory/transitory computer-readable storage media, such as RAM, ROM, EEPROM, EPROM, flash memory devices, or magnetic disks, and combinations thereof, and may include web storage of servers performing storage functions of the memory on the Internet. The memory 152 may store data 153 and instructions 154 necessary for the processor 151 to perform training of the artificial intelligence model.

For example, the training computing system 150 may include a model trainer 160 configured to train the machine learning models 120 and/or 140 stored in the user computing device 110 and/or the server computing system 130 by using various training or learning techniques such as backpropagation of an error.

For example, the model trainer 160 may perform updating one or more parameters of the machine learning models 120 and/or 140 for the high-resolution image analysis-based diagnosis response provision service based on a defined loss function by a backpropagation scheme.

In some implementation examples, the performance of the backpropagation of the error may include performing truncated backpropagation through time. The model trainer 160 may perform multiple generalization techniques (for example, weight reduction, drop-out, and/or knowledge distillation) in order to enhance a generalization capability of the trained machine learning models 120 and/or 140.

For example, the model trainer 160 may train the machine learning models 120 and/or 140 based on a series of training data 161. Herein, the training data 161 may include, for example, different formats of data such as an image, an audio, and/or text.

Examples of image type data which may be used may include a video frame, LiDAR point cloud, an X-ray image, a computer tomography scan, a hyperspectral image, and/or various other types of images.

The training data 161 may be provided by the user computing device 110 and/or the server computing system 130. When the training computing device 150 trains the machine learning models 120 and/or 140 with respect to specific data of the user computing device 110, the machine learning models 120 and/or 140 may be characterized as a personalized model.

In addition, the model trainer 160 includes a computer logic utilized to provide a desired function.

Further, the model trainer 160 may be implemented as hardware, firmware, and/or software controlling a universal processor. In one implementation example, the model trainer 160 may include a program file stored in a storage device, and may be loaded to the memory 152 and executed by one or more processors 151. In another implementation example, the model trainer 160 includes one or more sets of computer-executable data 153 and instructions 154 stored in executable by a tangible computer-readable storage medium such as a RAM hard disk or an optical or magnetic medium.

The network 170 includes a 3rd Generation Partnership Project (3GPP) network, a Long Term Evolution (LTE) network, a World Interoperability for Microwave Access (WIMAX) network, Internet, a Local Area Network (LAN), Wireless Local Area Network (LAN), a Wide Area Network (WAN), a Personal Area Network (PAN), a Bluetooth network, a satellite broadcasting network, an analog broadcasting network, and/or a Digital Multimedia Broadcasting (DMB) network, but is not limited thereto.

In general, communication through the network 170 may be performed through various communication protocols (for example, TCP/IP, HTTP, SMTP, and/or FTP), encoding or formats (for example, HTML and/or XML), and/or protective schemas (for example, VPN, secure HTTP, and/or SSL) by using any type of wired and/or wireless communication.

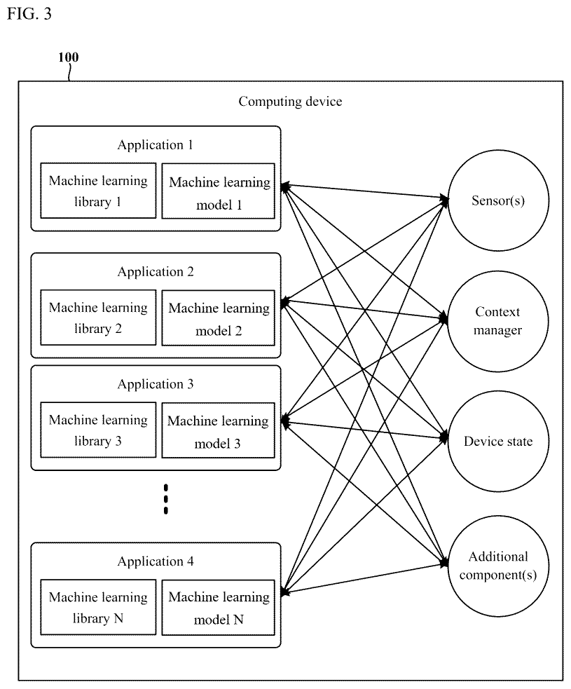

FIG. 3 is a block diagram illustrating the computing device 100 implementing a high-resolution image analysis-based diagnosis response provision service according to an embodiment.

Referring to FIG. 3, the computing device 100 included in one or more of the user computing device 110, the server computing system 130, and the training computing system 150 includes a plurality of applications (for example, application 1 to application N). Each application may include a machine learning library and at least one machine learning model. For example, the applications may include an image processing application (for example, an application for detection, classification and/or segmentation), a text messaging application, an e-mail application, a dictation application, a virtual keyboard application, a browser application, and a chat-bot application.

In an embodiment, the computing device 100 may include the model trainer 160 for training the artificial intelligence model, and may store and operate the trained artificial intelligence model to provide output data according to predetermined input data.

Each application of the computing device 100 may communicate with another or other components of the computing device 100, for example, but not limited to, one or more sensors, a context manager, a device state component, and/or additional components. In an embodiment, each application may communicate with each device component using an API (for example, a public API). In an embodiment, the API used by each application may be specific to the relevant application.

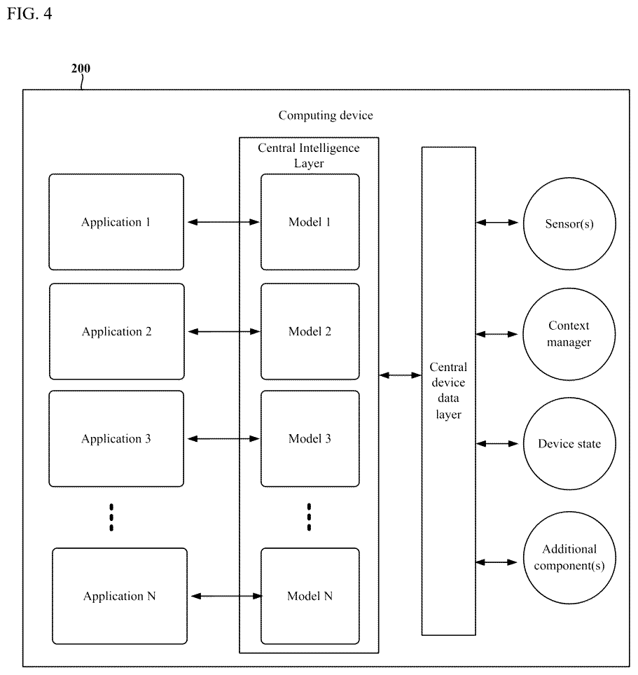

FIG. 4 is a block diagram illustrating a computing device 200 implementing a high-resolution image analysis-based diagnosis response provision service according to an embodiment.

Referring to FIG. 4, the computing device 200 includes a plurality of applications (for example, application 1 to application N). Each application is in communication with a central intelligence layer. For example, the applications may include an image processing application, a text messaging application, an e-mail application, a dictation application, a virtual keyboard application, and a browser application. In an embodiment, each application may communicate with the central intelligence layer (and model(s) stored therein) using an API (for example, a common API across all applications).

In addition, the central intelligence layer may include a plurality of machine learning models. For example, as illustrated in FIG. 4, a respective machine learning model and at least some thereof may be provided for each application and managed by the central intelligence layer. In other implementations, two or more applications may share a single machine leaning model. For example, in some implementations, the central intelligence layer may provide a single model for all of the applications. In some implementations, the central intelligence layer may be included within an operating system of the computing device 200 or implemented differently.

The central intelligence layer may communicate with a central device data layer. The central device data layer may be a centralized data storage for the computing device 200. As illustrated in FIG. 4, the central device data layer may communicate with another or other components of the computing device 200, such as, for example, one or more sensors, a context manager, a device state component, and/or additional components. In some implementations, the central device data layer may communicate with each device component using an API (for example, a private API).

The embodiments discussed herein make reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein may be implemented using a single device or component or a plurality of devices or components working in combination. Databases and applications may be implemented on a single system or distributed across a plurality of systems. Distributed components may operate sequentially or in parallel.

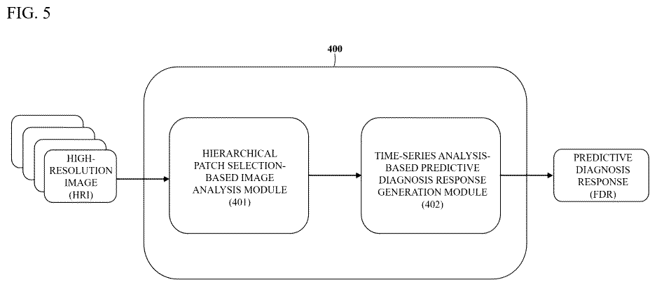

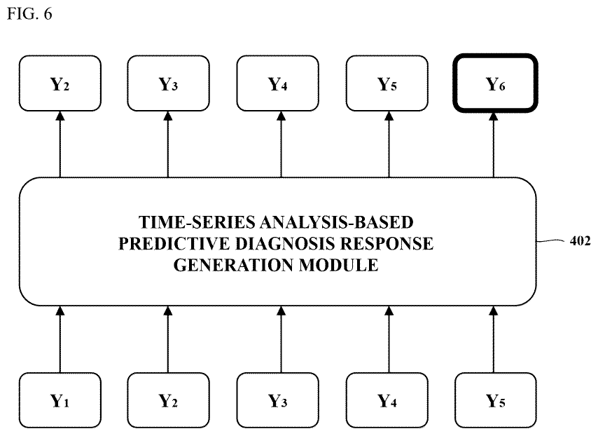

FIG. 5 is a block diagram illustrating a computing device 400 implementing a high-resolution image analysis-based diagnosis response provision service according to an embodiment. FIG. 6 is a conceptual diagram illustrating the generation of a predictive diagnosis response by a time-series analysis-based predictive diagnosis response generation module 402 according to an embodiment.

Referring to FIG. 5, the computing device 400 included in one or more of the user computing device 110, the server computing system 130, and/or the training computing system 150 may include a hierarchical patch selection-based image analysis module 401 configured to perform individual analysis on a high-resolution image (HRI) and a time-series analysis-based predictive diagnosis response generation module 402 configured to receive a plurality of image analysis results from the hierarchical patch selection-based image analysis module 401 and output a predictive diagnosis response (FDR) for a plurality of HRIs.

The hierarchical patch selection-based image analysis module 401 may perform image analysis based on hierarchical patch selection on each of the plurality of HRIs and output a plurality of analysis prediction values.

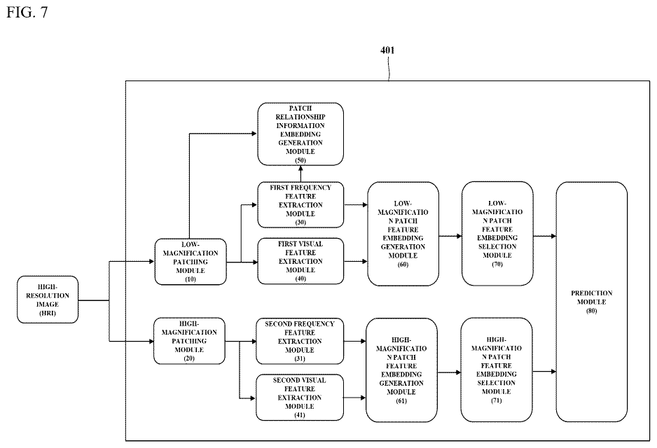

A more detailed description of the hierarchical patch selection-based image analysis module 401 will be described below with reference to FIGS. 7 to 11.

The time-series analysis-based predictive diagnosis response generation module 402 may receive a plurality of analysis prediction values output from the hierarchical patch selection-based image analysis module 401 as an input, analyze the analysis prediction values as time-series data, and output a final predictive diagnosis response (FDR) for the plurality of HRIs.

The time-series analysis-based predictive diagnosis response generation module 402 goes or performs beyond static predictions for a single image and comprehensively grasps temporal change patterns in data across a plurality of points in time, thereby increasing prediction accuracy and reliability.

The time-series analysis-based predictive diagnosis response generation module 402 may align the plurality of analysis prediction values by the order of point in time and generate a positional embedding for each prediction value that includes time information at a point in time when the image was photographed, thereby configuring time-series data. Based on this time-series data, the time-series analysis-based predictive diagnosis response generation module 402 may train a time-series analysis model, such as a recurrent neural network (RNN), a long short-term memory (LSTM), or a Transformer.

For example, referring to FIG. 6, the transformer-based time-series analysis-based predictive diagnosis response generation module 402 may receive a plurality of sequential analysis prediction values from Y1 to Y5 as the time-series data, and learn the temporal pattern and long-term dependency of the relevant data through a self-attention mechanism. In this learning process, the time-series analysis-based predictive diagnosis response generation module 402 may be trained to compute a predictive diagnosis response Y6 for the next point in time.

The predictive diagnosis response (FDR) provided by the time-series analysis-based predictive diagnosis response generation module 402 may include precise information reflecting temporal changes, such as a disease progression, a grade of the disease, a likelihood of recurrence of the disease, and a future change trend of the disease for tissue pathology images, development progression in a specific region, changes in forest area, or coastline erosion trends for satellite images, occurrence of minute defects in products for product production line images, abnormal movement patterns and the possibility of invasion in a specific region for surveillance camera images, and growth status of crops or spread of pests for agricultural field images.

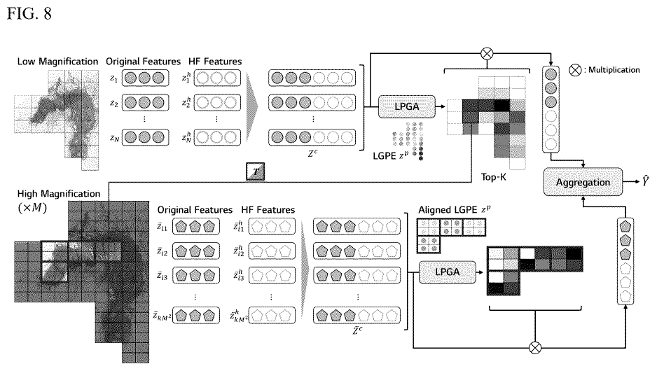

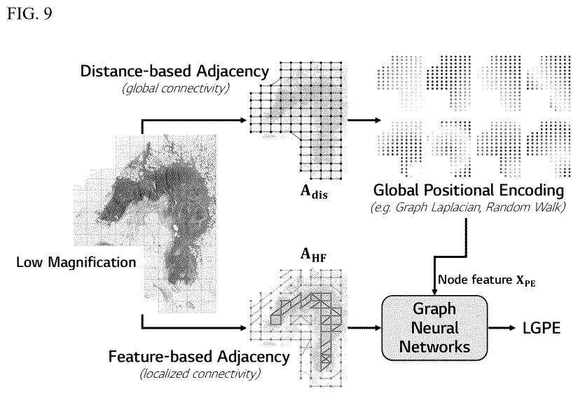

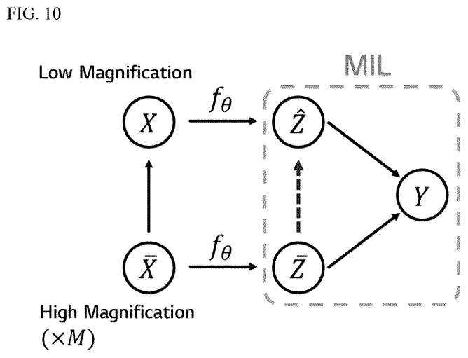

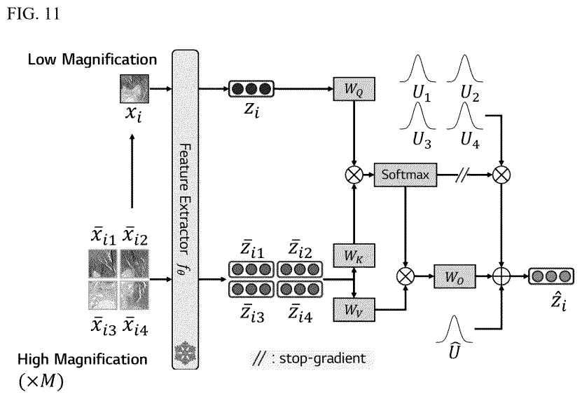

FIG. 7 is a block diagram illustrating an exemplary configuration of the hierarchical patch selection-based image analysis module 401 utilized to implement a high-resolution image analysis-based diagnosis response provision service according to an embodiment. FIG. 8 illustrates an exemplary configuration of a spectral analysis-based multi-resolution analysis architecture for a high-resolution image according to an embodiment. FIG. 9 is a conceptual diagram for illustrating a method for extracting positional information and characteristic relationship information between a plurality of patches of a high-resolution image according to an embodiment. FIG. 10 is a conceptual diagram illustrating a framework for a multi-resolution integrated model that reflects causal relationships between multiple magnifications according to an embodiment. FIG. 11 is a configuration diagram illustrating a framework for a multi-resolution integrated model that reflects causal relationships between multiple magnifications according to an embodiment.

Referring to FIG. 7, the hierarchical patch selection-based image analysis module 401 may include a low-magnification patching module 10, a high-magnification patching module 20, a first frequency feature extraction module 30, a first visual feature extraction module 40, a second frequency feature extraction module 31, a second visual feature extraction module 41, a patch relationship information embedding generation module 50, a low-magnification patch feature embedding generation module 60, a high-magnification patch feature embedding generation module 61, a low-magnification patch feature embedding selection module 70, a high-magnification patch feature embedding selection module 71, and a prediction module 80.

The low-magnification patching module 10 may receive a high-resolution image (HRI) and process the HRI into a low-magnification image, such as 10-fold (10×) or 5-fold (5×). The low-magnification patching module 10 may efficiently secure data on low-magnification images necessary to grasp the overall context and structure among the vast amount of information in the HRI.

The low-magnification patching module 10 may segment the generated low-magnification image into the plurality of patches using a method such as a sliding window. However, the present disclosure is not limited thereto, and various methods other than a method of utilizing the sliding window may be adopted as a method of segmenting the low-magnification image into the plurality of patches.

The low-magnification patching module 10 generates patch embeddings for each of the plurality of segmented patches, and then these patch embeddings may be provided as an input to the first visual feature extraction module 40 and the first frequency feature extraction module 30. Through this process, rich contextual information at low magnification may be transferred to the next analysis stage.

The high-magnification patching module 20 may receive the HRI and process the HRI into a high-magnification image, such as 20-fold (20×) or 10-fold (10×). The high-magnification patching module 20 may efficiently secure high-magnification image data necessary to grasp subtle morphological clues at the cellular level from the vast amount of information in the HRI.

The high-magnification patching module 20 may segment the generated high-magnification image into the plurality of patches using a method such as a sliding window. However, the present disclosure is not limited thereto, and various methods other than a method of utilizing the sliding window may be adopted as a method of segmenting the high-magnification image into the plurality of patches.

The high-magnification patching module 20 may generate patch embeddings for each of the plurality of segmented patches, and then these patch embeddings may be provided as an input to the second visual feature extraction module 41 and the second frequency feature extraction module 31. Through this process, subtle information at high-magnification may be transferred to the next analysis stage.

The first frequency feature extraction module 30 may receive the plurality of patches segmented by the low-magnification patching module 10 and extract frequency feature embeddings. Thus, the first frequency feature extraction module 30 may capture subtle, diagnostically significant morphological clues in high-resolution images.

The first frequency feature extraction module 30 performs a Fourier transform on the input patch to acquire frequency domain data, and then filters the frequency domain data of the input patch to remove low-frequency components from the frequency domain data and separate only the high-frequency components (HF features) necessary for diagnosis.

The high-frequency components of the patch contain subtle detailed information, such as cellular structures and tissue boundaries, as well as sharp transitions, making the same very important for pathological diagnosis. This contributes to improving model accuracy and reliability by capturing diagnostic clues that may not be able to be found in low-magnification images or may easily be missed in low-magnification images.

Finally, the first frequency feature extraction module 30 performs an inverse Fourier transform on the separated high-frequency components and inputs the data generated by performing the inverse Fourier transform to a pre-trained vision encoder (fᵥ), thereby generating a frequency feature embedding (z^{h}).

For example, referring to FIG. 8, the first frequency feature extraction module 30 may receive a plurality of patches for the low-magnification image, and generate a plurality of first frequency feature embeddings

$$( {z}_{i}^{h} )$$

of high-frequency components corresponding to the plurality of patches (i is a positive integer from 1 to N). The plurality of first frequency feature embeddings

$$( {z}_{i}^{h} )$$

may be provided as inputs to the low-magnification patch feature embedding generation module 60 in a subsequent stage.

The process of extracting frequency feature embeddings by receiving the plurality of patches of the first frequency feature extraction module 30 may be performed based on Equations (1) and (2) below.

$$\tilde{x} = ℱ ( x ) , \qquad Equation ( l )$$

$${\tilde{x}}^{h} = t ( \tilde{x} ; r )$$

(wherein, x: patch embedding, F(·): Fourier transform function, \tilde{x}: Fourier transform patch embedding, \tilde{x}^{h}: high-frequency Fourier transform patch embedding, t(\tilde{x}; r): thresholding function, and r: hyperparameter radius)

$${z}^{h} = {f}_{v} ( {ℱ}^{- 1} ( {\tilde{x}}^{h} ) ) \qquad Equation ( 2 )$$

(wherein, fᵥ(·): vision encoding function, F⁻¹(·): Fourier transform function, \tilde{x}^{h}: high-frequency Fourier transform patch embedding, and z^{h}: high-frequency patch embedding)

The first visual feature extraction module 40 may receive the plurality of patches segmented by the low-magnification patching module 10 and extract original features. The original features may refer to the visual features of an image, and the first visual feature extraction module 40 may capture the overall visual information of the image and provide a basic feature representation for analysis of the subsequent stage.

The first visual feature extraction module 40 may extract visual features using a pre-trained vision encoder (for example, ResNet), which may compress the visual patterns of the input patches and transform the compressed visual patterns of the input patches into latent representations. During this process, the parameters of the vision encoder may be used in a frozen state to reduce the computational burden caused by the large number of patches in a high-resolution image.

For example, referring to FIG. 8, the first visual feature extraction module 40 may receive a plurality of patches for the low-magnification image, and generate a plurality of first visual feature embeddings (zi) corresponding to the original features of the plurality of patches. The plurality of first visual feature embeddings (zi) may be provided as inputs of the low-magnification patch feature embedding generation module 60 in a subsequent stage.

The second frequency feature extraction module 31 may receive the plurality of patches segmented by the high-magnification patching module 20 and extract frequency feature embeddings.

Similar to the first frequency feature extraction module 30, the second frequency feature extraction module 31 may separate only high-frequency components through the Fourier transform and thresholding or filtering of the patches, and generate the final frequency feature embeddings through the inverse Fourier transform and the vision encoder.

For example, referring to FIG. 8, the second frequency feature extraction module 31 may receive a plurality of patches for a high-magnification image, and generate a plurality of second frequency feature embeddings

$$( {\overline{z}}_{ij}^{h} )$$

of high-frequency components corresponding to the plurality of patches (j is a positive integer from 1 to M², and M is a positive integer). The plurality of second frequency feature embeddings

$$( {\overline{z}}_{ij}^{h} )$$

may be provided as inputs of the high-magnification patch feature embedding generation module 61 in a subsequent stage. The second frequency feature extraction module 31 may perform the required function as specified in the description of the first frequency feature extraction module 30.

The second visual feature extraction module 41 may receive the plurality of patches segmented by the high-magnification patching module 20 and extract original features. The original features may refer to visual features of an image, and the second visual feature extraction module 41 may capture the overall visual information of the image and provide a basic feature representation for analysis in a subsequent stage.

For example, referring to FIG. 8, the second visual feature extraction module 41 may receive a plurality of patches for a high-magnification image, and generate a plurality of second visual feature embeddings (z̅ᵢⱼ) corresponding to the original features of the plurality of patches. The plurality of second visual feature embeddings (z̅ᵢⱼ) may be provided as inputs of the high-magnification patch feature embedding generation module 61 in a subsequent stage. The second visual feature extraction module 41 may perform the required function as specified in the description of the first visual feature extraction module 40.

Although the first frequency feature extraction module 30 and the second frequency feature extraction module 31 are illustrated as separate or independent modules in the exemplary embodiment of FIG. 8, the present disclosure is not limited thereto, and a single integrated frequency feature extraction module for performing the function of the both the first frequency feature extraction module 30 and the second frequency feature extraction module 31 may be implemented to extract frequency feature embeddings for both patches from the low-magnification patching module 10 and patches from the high-magnification patching module 20.

This integrated frequency feature extraction module may recognize magnification information (for example, 10×, 20×) of input patches and extract optimal frequency features for each patch. This improves system efficiency by increasing module resource utilization.

The patch relationship information embedding generation module 50 may encode geometric positional relationships and morphological similarities between patches based on the plurality of patches segmented by the low-magnification patching module 10.

For example, referring to FIG. 9, the patch relationship information embedding generation module 50 may generate each of an adjacency matrix (A_{dis}) based on distance information between patches and an adjacency matrix (A_{HF}) based on a similarity of frequency features, based on the plurality of patches from the low-magnification image. Using the same, a plurality of patch relationship information embeddings

$$( {z}_{i}^{P} )$$

corresponding to the LGPE may be generated.

Herein, the reason that the patch relationship information embedding generation module 50 generates the plurality of patch relationship information embeddings

$$( {z}_{i}^{P} )$$

based on low-magnification images is to prevent significant increases in computational complexity and memory usage due to the large number of patches when based on high-magnification images.

In detail, the process of computing the plurality of patch relationship information embeddings

$$( {z}_{i}^{P} )$$

includes matrix arithmetic operations (for example, eigenvalue decomposition for a graph Laplacian), which, as described below, rapidly increase in computational complexity as the number of patches (N) increases. Accordingly, by computing the plurality of patch relationship information embeddings

$$( {z}_{i}^{P} )$$

from low-magnification images with a significantly smaller number of patches and aligning and reusing the same with high-magnification patches, the need for calculating positional information and feature relationship position encoding for the plurality of patches in the high-magnification image may be eliminated, thereby increasing or maximizing efficiency.

The patch relationship information embedding generation module 50 learns the structural relationships between low-magnification image patches and generates the LGPE necessary to effectively evaluate the importance of patches in subsequent stages and resolve the multi-resolution dilemma.

The patch relationship information embedding generation module 50 may utilize two types of adjacency matrices to generate the plurality of patch relationship information embeddings (z^{P}).

First, a distance-based adjacency matrix (A_{dis}) models the global connectivity between patches based on the Euclidean distances of the patches.

For example, the patch relationship information embedding generation module 50 considers the center points of the plurality of patches as nodes and computes the distances between these nodes to construct a graph. The distance-based adjacency matrix (A_{dis}) represents the connectivity between these nodes. A_{dis}(i, j) has a value of 1 when the j-th patch is within the top-k nearest patches of the i-th patch, and the distance therebetween does not exceed a predetermined distance threshold. Through this process, information about how patches are physically distributed and how close the patches are to each other may be encoded in the distance-based adjacency matrix (A_{dis}).

Furthermore, the distance-based adjacency matrix (A_{dis}) is used to eigen-decompose the graph Laplacian (Δ) to compute a plurality of eigenvectors (U) that encode the geometric locations of the patches. The eigenvector (U) has invariance to slide rotation, which is an important characteristic for high-resolution image analysis.

For example, according to Equation (3) below, the graph Laplacian (Δ) may be defined utilizing the distance-based adjacency matrix (A_{dis}) and the degree matrix (D_{dis}), and then eigen-decomposed to compute the plurality of eigenvectors (U) that encode the geometric locations of the patches. The plurality of eigenvectors (U) may be referred to as a plurality of positional feature vectors.

$$Δ = {I}_{n} - {D}_{dis}^{- 1 / 2} {A}_{dis} {D}_{dis}^{- 1 / 2} = {U}^{T} Λ U \qquad Equation ( 3 )$$

(wherein, Δ is the graph Laplacian, Iₙ is the identity matrix, D_{dis} is the degree matrix, A_{dis} is the distance-based adjacency matrix, U is the eigenvector matrix, and Λ is the eigenvalue matrix)

The degree matrix (D_{dis}) represents the degree of concatenation of each patch, and may be created by disposing the sum of the row or column elements of the adjacency matrix (A_{dis}) along the diagonal. In other words, the degree matrix (D_{dis}) is a symmetric matrix with values only on the diagonal elements and zeros on the rest of the elements of the matrix. The values of the diagonal elements correspond to the number of adjacent patches within a threshold distance among the top-k adjacent patches for the i-th patch.

The eigenvector matrix (U), obtained by eigenvector decomposition of the distance-based adjacency matrix (A_{dis}), encodes geometric positional information between patches. These eigenvectors serve as a key input for generating the plurality of patch relationship information embedding

$$( {z}_{i}^{P} ) .$$

These eigenvectors are generated based on the distance-based adjacency matrix (A_{dis}), which is generated based on the Euclidean distance between patches, and has important properties such as invariance to slide rotation for high-resolution images.

Second, a frequency feature-based adjacency matrix (A_{HF}) performs or goes beyond Euclidean distance and models local connectivity between patches based on the similarity (for example, cosine similarity) between a plurality of high-frequency features of the plurality of patches.

This allows for the capture of similarities in subtle morphological patterns important for diagnosis, such as adipocytes and cytoplasm.

For example, the patch relationship information embedding generation module 50 computes the similarity between a plurality of frequency feature embeddings for the plurality of patches in the low-magnification image and generates the frequency feature-based adjacency matrix (A_{HF}) based on the similarity information between a plurality of frequency embeddings and the distance information between the plurality of patches in the low-magnification image.

This process goes or performs beyond simply determining connectivity based on distance alone and models local connectivity by considering the similarity of subtle morphological patterns between patches.

Specifically, according to Equation (4) below, the connectivity (e_{i,j}) between the i-th and j-th patches among the plurality patches of the low-magnification image, corresponding to an element of the frequency feature-based adjacency matrix (A_{HF}), has a value of 1 when both conditions are met.

First, the j-th patch included in the plurality of patches of the low-magnification image is required to be one of the top k patches with the most similar frequency feature embeddings to the i-th patch. Second, the distance d(i,j) between the i-th and j-th patches is required not to exceed a specific distance threshold (τ). Based on the connectivity information generated through this process, the frequency feature-based adjacency matrix (A_{HF}) may be constructed.

$${e}_{i , j} = \{ \begin{matrix}1 , & if d ( i , j ) \leq τ and j ϵ top - k ( {S}_{i , :} ) \\ 0 , & otherwise\end{matrix}$$

The patch relationship information embedding generation module 50 may input a compressed positional feature matrix (X_{PE}) and the frequency feature-based adjacency matrix (A_{HF}), generated based on a portion selected according to predetermined conditions from among the acquired plurality of eigenvectors (U), into a graph neural network (GNN) to ultimately compute the plurality of patch relationship information embeddings

$$( {z}_{i}^{P} )$$

corresponding to the LGPE.

The GNN combines these two pieces of information to generate the plurality of patch relationship information embeddings

$$( {z}_{i}^{P} )$$

that reflect both the positional and morphological similarities of the patches, which may be provided as an input to the low-magnification patch feature embedding selection module 70 and the high-magnification patch feature embedding selection module 71 that perform an LGPE-aware gated attention (LPGA) arithmetic operation, which is a patch selection mechanism, in a subsequent stage.

In detail, using Equation (5) below, the patch relationship information embedding generation module 50 may generate a compressed positional feature matrix (X_{PE}) based on one or more of the plurality of eigenvectors (U) selected according to predetermined conditions.

X_{PE}=tanh(U_{[:,:k}_{_{PE}}_{]}W) Equation (5)
(wherein, U: eigenvector matrix, k_{PE}: number of selected eigenvectors, and W: learnable weight matrix.)

Then, the GNN performs neural network operations to learn a graph structure modeling the relationships between the plurality of patches, according to Equation (6) below, based on the input compressed positional feature matrix (X_{PE}) and frequency feature-based adjacency matrix (A_{HF}), thereby generating the plurality of patch relationship information embedding

$$( {z}_{i}^{P} ) .$$

z^{P}=GNN(X_{PE},A_{HF}) Equation (6)

GNN(·) may be implemented by, for example, a graph convolutional network (GCN) operation method that performs matrix-multiplication on the compressed positional feature matrix (X_{PE}) and the frequency feature-based adjacency matrix (A_{HF}), and then matrix-multiplies the same by a predetermined weight matrix (W) to compute the plurality of patch relationship information embeddings

$$( {z}_{i}^{P} ) .$$

However, the present disclosure is not limited thereto, and GNN(·) may be implemented by various operation methods in addition to the GCN operation method, such as a GraphSAGE operation method that samples and aggregates the features of neighboring nodes, a GAT (Graph Attention Networks) operation method that uses an attention mechanism to give different weights to neighboring nodes, or a GIN (Graph Isomorphism Network) operation method that preserves the unique characteristics of the graph structure by summing the node features and neighboring features.

The low-magnification patch feature embedding generation module 60 may generate a plurality of first patch feature embeddings

$$( {z}_{i}^{c} )$$

by integrating the plurality of first visual feature embeddings (zᵢ) generated by the first visual feature extraction module 40 and the plurality of first frequency feature embeddings

$$( {z}_{i}^{h} )$$

generated by the first frequency feature extraction module 30. Referring to FIG. 6, the plurality of first patch feature embeddings

$$( {z}_{i}^{c} )$$

may correspond to a first patch feature integration matrix (Z^{c}).

The low-magnification patch feature embedding generation module 60 combines two different perspectives, visual information and frequency information, for the plurality of patches of the low-magnification image, thereby enabling a high-resolution image analysis model trained based thereon to understand the features of the patch more richly and comprehensively.

The low-magnification patch feature embedding generation module 60 may use a method of concatenating the plurality of first visual feature embeddings (zᵢ) and the plurality of first frequency feature embeddings

$$( {z}_{i}^{h} )$$

to generate the plurality of first patch feature embeddings

$$( {z}_{i}^{c} ) .$$

The plurality of first patch feature embeddings

$$( {z}_{i}^{c} )$$

generated through this integration include both the general visual pattern of the patch and the subtle morphological clues (high-frequency components) information that are important for diagnosis. The plurality of first patch feature embeddings

$$( {z}_{i}^{c} )$$

generated as such may be provided as an input to the low-magnification patch embedding selection module 70.

The high-magnification patch feature embedding generation module 61 may generate a plurality of second patch feature embeddings

$$( {\overline{z}}_{ij}^{c} )$$

by integrating the plurality of second visual feature embeddings (z̅ᵢⱼ) generated by the second visual feature extraction module 41 and the plurality of second frequency feature embeddings

$$( {\overline{z}}_{ij}^{h} )$$

generated by the second frequency feature extraction module 31. Referring to FIG. 6, the plurality of second patch feature embeddings

$$( {\overline{z}}_{ij}^{c} )$$

may correspond to a second patch feature integration matrix (Z̅^{c}).

The high-magnification patch feature embedding generation module 61 combines two different perspectives, visual information and frequency information, for the plurality of patches of a high-magnification image, thereby enabling the high-resolution image analysis model trained based thereon to understand the features of the patch more richly and comprehensively. A more detailed description of the function of the high-magnification patch feature embedding generation module 61 follows the description of the function of the patch feature embedding generation module 60.

While the low-magnification patch feature embedding generation module 60 and the high-magnification patch feature embedding generation module 61 are illustrated as separate modules in FIG. 7, the present disclosure is not limited to thereto, and a single module may be implemented to generate patch feature embeddings for both magnifications. In this configuration, the module may recognize the magnification information of the input patch and generate feature embeddings optimized for each magnification.

The low-magnification patch feature embedding selection module 70 is a key module that implements a hierarchical patch selection method in the learning data processing process for training the high-resolution image analysis model according to various embodiments of the present disclosure.

The low-magnification patch feature embedding selection module 70 may efficiently select some of the patches corresponding to the uppermost importance for diagnosis by utilizing an LPGA mechanism for the plurality of first patch feature embeddings

$$( {z}_{i}^{c} )$$

generated by the low-magnification patch feature embedding generation module 60. The LPGA may mean an arithmetic operation that receives as an input a patch integrated embedding

$$( {z}_{i}^{PE} )$$

for each patch of the low-magnification image and computes a predetermined weight for selecting one or more of the plurality of first patch feature embeddings

$$( {z}_{i}^{c} ) .$$

First, the patch integrated embedding

$$( {z}_{i}^{PE} )$$

for each patch of the low-magnification image may be generated by integrating the visual feature embedding (zᵢ), frequency feature embedding

$$( {z}_{i}^{h} ) ,$$

and patch relationship information embedding

$$( {z}_{i}^{P} )$$

of each patch of the low-magnification image. In this configuration, the patch integrated embedding

$$( {z}_{i}^{PE} )$$

for each patch of the low-magnification image may be generated by concatenating the visual feature embedding (zᵢ), frequency feature embedding

$$( {z}_{i}^{h} ) ,$$

and patch relationship information embedding

$$( {z}_{i}^{P} )$$

for each patch of the low-magnification image.

Thereafter, the low-magnification patch feature embedding selection module 70 may compute a plurality of attention weights (αi) based on the patch integrated embedding

$$( {z}_{i}^{PE} )$$

corresponding to each patch of the low-magnification image according to Equation (7) below. This weight indicates how the corresponding patch contributes to the final prediction.

$${α}_{i} = \frac{exp ( {ω}^{T} ( tanh ( V {z}_{i}^{PE} ) ⊙ η ( U {z}_{i}^{PE} ) ) )}{{\sum }_{j = 1}^{N} exp ( {ω}^{T} ( tanh ( V {z}_{i}^{PE} ) ⊙ η ( U {z}_{i}^{PE} ) ) )} \qquad Equation ( 7 )$$

(wherein, V, U: learnable weight matrices, η(·): sigmoid function)

Thereafter, the low-magnification patch feature embedding selection module 70 may select k embeddings from among the plurality of first patch feature embeddings

$$( {z}_{i}^{c} ) ,$$

corresponding to the top k weights (k is a positive integer) among the plurality of weights computed. The selected k first patch feature embeddings

$$( {z}_{i}^{c} )$$

may be provided as an input to the prediction module 80, which is a subsequent stage. For example, k is a value that may be selected in various ways depending on the type of high-resolution image, and may be set to various numbers such as 16, 20, and 300.

The high-magnification patch feature embedding selection module 71 may efficiently select a portion corresponding to patches with a high degree of diagnostic importance by utilizing the LPGA mechanism for the plurality of second patch feature embeddings

$$( {\overline{z}}_{ij}^{c} )$$

generated by the high-magnification patch feature embedding generation module 61. The LPGA may mean an arithmetic operation that takes as an input the patch integrated embedding

$$( {\overline{z}}_{ij}^{PE} )$$

for each patch of the high-magnification image and computes a predetermined weight for selecting one or more of the plurality of second patch feature embeddings

$$( {\overline{z}}_{ij}^{c} ) .$$

First, the patch integrated embedding

$$( {\overline{z}}_{ij}^{PE} )$$

for each patch of the high-magnification image may be generated by integrating the visual feature embedding (z̅ᵢⱼ), frequency feature embedding

$$( {\overline{z}}_{ij}^{h} ) ,$$

and patch relationship information embedding

$$( {z}_{i}^{P} )$$

of each patch of the high-magnification image. In this configuration, the patch integrated embedding

$$( {\overline{z}}_{ij}^{PE} )$$

for each patch of the high-magnification image may be generated by concatenating the visual feature embedding (z̅ᵢⱼ), frequency feature embedding

$$( {\overline{z}}_{ij}^{h} ) ,$$

and patch relationship information embedding

$$( {z}_{i}^{P} )$$

for each patch of the high-magnification image.

As such, when the patch integrated embedding

$$( {\overline{z}}_{ij}^{PE} )$$

for each patch of the high-magnification image is generated, the patch relationship information embedding

$$( {z}_{i}^{P} )$$

computed based on the low-magnification image may be utilized as is. This eliminates the computational need for generating the patch relationship information embeddings

$$( {z}_{i}^{P} )$$

for the plurality of patches in a massive amount of high-magnification images, while applying consistent geometric information to heterogeneous patches. This may improve the computational efficiency of high-resolution analysis models and contributes to enhanced prediction performance by maintaining the overall contextual information to which the plurality of patches belong.

Thereafter, the high-magnification patch feature embedding selection module 71 may compute the plurality of attention weights (αij) based on the patch integrated embedding

$$( {\overline{z}}_{ij}^{PE} )$$

corresponding to each patch of the high-magnification image, according to Equation (7) above. These weights indicate how significantly the corresponding patch contributes to the final prediction.

Thereafter, the high-magnification patch feature embedding selection module 71 may select k×M² embeddings corresponding to the top k×M² (M is the relative magnification of high magnification to low magnification) weights among the plurality of weights computed from among the plurality of second patch feature embeddings

$$( {\overline{z}}_{ij}^{c} ) .$$

The selected k×M² second patch feature embeddings

$$( {\overline{z}}_{ij}^{c} )$$

may be provided as an input to the prediction module 80, which is a subsequent stage.

As such, when the high-magnification patch feature embedding selection module 71 selects k×M² embeddings from among the plurality of second patch feature embeddings

$$( {\overline{z}}_{ij}^{c} ) ,$$

the areas occupied by the patches corresponding to the k first patch feature embeddings

$$( {z}_{i}^{c} )$$

selected by the low-magnification patch feature embedding selection module 70 on the low-magnification image may match the areas occupied by the patches corresponding to the k×M² second patch feature embeddings

$$( {\overline{z}}_{ij}^{c} )$$

selected by the high-magnification patch feature embedding selection module 71 on the high-magnification image.

In other word, the high-resolution image analysis model trained based on the data generated through this process may effectively integrate information from both magnifications by analyzing areas determined to be diagnostically significant in the low-magnification image in the same manner in the high-magnification image.

The prediction module 80 may be trained to output a prediction value at the overall level of the high-resolution image by synthesizing features selected from each of the low-magnification image and the high-magnification image.

The prediction module 80 acts as an aggregator that integrates features extracted from the plurality of patches in a multi-instance learning (MIL) framework based on a method of segmenting the high-resolution image into the plurality of patches according to an embodiment of the present disclosure, and may be trained to provide a final diagnosis response for the entire high-resolution image.

First, the prediction module 80 may compute a weighted sum for the k first patch feature embeddings

$$( {z}_{i}^{c} )$$

selected by the low-magnification patch feature embedding selection module 70. This weighted sum may be computed based on k attention weights (αi) selected from among the plurality of attention weights (αi) computed based on the patch integrated embeddings

$$( {z}_{i}^{PE} )$$

corresponding to each patch of the low-magnification image. By applying a g(·) function (linear head) to this weighted sum, a bag-level representation g(Z^{c}) that integrates the k first patch feature embeddings

$$( {z}_{i}^{c} )$$

for the low-magnification image may be generated.

In addition, the prediction module 80 may compute a weighted sum for the k×M² second patch feature embeddings

$$( {\overline{z}}_{ij}^{c} )$$

selected by the high-magnification patch feature embedding selection module 71. This weighted sum may be computed based on k×M² attention weights (αij) selected from among the plurality of attention weights (αij) computed based on the patch integrated embeddings

$$( {\overline{z}}_{ij}^{PE} )$$

corresponding to each patch of the high-magnification image. By applying the g(·) function (linear head) to this weighted sum, the bag level representation g(Z̅^{c}) that integrates the k×M² second patch feature embeddings

$$( {\overline{z}}_{ij}^{c} )$$

for the high-magnification image may be generated.

Finally, the prediction module 80 may perform a predetermined arithmetic operation, including the f(·) function, on these two bag level representations (g(Z^{c}), g(Z̅^{c})) to output a prediction value (Ŷ) at the overall level of the final high-resolution image.

This f(·) function may be configured or designed to combine the two bag level representations and perform a linear transformation for final classification. The prediction module 80 may be trained to reduce or minimize the error between the final prediction value (Ŷ) and the ground truth data.

As such, the prediction module 80 is configured to output the final prediction value (Ŷ) by utilizing the weighted sum of the selected k first patch feature embeddings

$$( {z}_{ij}^{c} )$$

and the weighted sum of the selected k×M² second patch feature embeddings

$$( {\overline{z}}_{ij}^{c} ) ,$$

thereby comprehensively reflecting both the broad contextual information of the low-magnification image based on the importance of each patch and the subtle clue information of the high-magnification image, thereby improving the prediction performance of multi-resolution image analysis and effectively alleviating the multi-resolution dilemma.

However, the present disclosure is not limited thereto, and the prediction module 80 may be configured to output the final prediction value (Ŷ) by performing a predetermined arithmetic operation on the selected k first patch feature embeddings

$$( {z}_{i}^{c} )$$

and the selected k×M² second patch feature embeddings

$$( {\overline{z}}_{ij}^{c} ) .$$

In such an embodiment, instead of simply outputting the final prediction value (Ŷ) using the weighted sum of the plurality of patch feature embeddings from the low-magnification image and the weighted sum of the plurality of patch feature embeddings from the high-magnification image, the prediction module 80 may apply a multi-resolution aggregation with causal perspective (MAC) method according to the framework of the multi-resolution integrated model that reflects the causal relationship between multiple magnifications, as illustrated in FIGS. 8 and 9, which explicitly models the causal relationship between multiple magnifications.

According to the MAC method, the prediction module 80 may utilize a cross-attention arithmetic operation based on the selected k first patch feature embeddings

$$( {z}_{i}^{c} )$$

and the selected k×M² second patch feature embeddings

$$( {\overline{z}}_{ij}^{c} )$$

to model the causal relationship between multiple magnifications.

This arithmetic operation uses the selected k first patch feature embeddings

$$( {z}_{i}^{c} )$$

as queries (qᵢ) and k×M² second patch feature embeddings

$$( {\overline{z}}_{ij}^{c} )$$

corresponding thereto as keys (kᵢⱼ) and values (vᵢⱼ) according to Equations (8) to (10) below. Through this process, k new low-magnification dependent feature embeddings

$$( {z}_{i}^{c ′} )$$

that are causally influenced by the features of the high-magnification image may be generated.

$${a}_{ij} = \frac{{q}_{i} {k}_{ij}^{T}}{\sqrt{{d}_{k}}} \qquad Equation ( 8 )$$

$${α}_{ij} = \frac{exp ( {a}_{ij} )}{{\sum }_{j = 1}^{{M}^{2}} exp ( {a}_{ij} )} \qquad Equation ( 9 )$$

$${z}_{i}^{c ′} = ( {{\sum }_{j = 1}}^{{M}^{2}} {α}_{ij} {v}_{ij} ) {W}_{O} \qquad Equation ( 10 )$$

(wherein, Wₒ is a learnable parameter)

Herein, the causal influence of the features in the high-magnification image refers to the influence of high-magnification features on low-magnification features in a latent space, such as down-sampling of the high-magnification image in an image space to generate the low-magnification image. The MAC method may overcome the limitations of conventional simple associative learning by explicitly modeling the structural and hierarchical relationships.

Furthermore, the prediction module 80 may reinforce the causal modeling by introducing unexplained factors of a latent space down-sampling process as noise (U, Û).

For example, the prediction module 80 may generate k integrated low-magnification dependent feature embeddings

$$( {\hat{z}}_{i}^{c} )$$

by adding a weighted sum of a plurality of patch-level noises (U) for the plurality of patches of the high-magnification image and the overall image-level noise (Û) for the high-resolution image to each of the k low-magnification dependent feature embeddings

$$( {z}_{i}^{c ′} ) ,$$

according to Equation (11) below.

The weights used in the weighted sum of patch-level noise (U) may be the same as the weights generated in the cross-attention arithmetic operation process to compute the k low-magnification dependent feature embeddings

$$( {z}_{i}^{c ′} ) .$$

$${\hat{z}}_{i}^{c} = {z}_{i}^{c ′} + {{\sum }_{j = 1}}^{{M}^{2}} sg ( {α}_{ij} ) {U}_{j} + \hat{U} \qquad Equation ( 11 )$$

Finally, the prediction module 80 may be configured to output a prediction value (Ŷ) at an image-wide level based on the result of integrating the k×M² second patch feature embeddings

$$( {\overline{z}}_{ij}^{c} )$$

and the k integrated low-magnification dependent feature embeddings

$$( {\hat{z}}_{i}^{c} ) .$$

As such, the high-resolution image analysis model may improve prediction performance and reliability by learning causal dependencies beyond simple associations between low- and high-magnification features.

### —Method for Training High-Resolution Image Analysis Model (S100)

A method S100 for training the high-resolution image analysis model may efficiently select patches and perform final predictions by utilizing information that combines frequency area information and positional information through spectral analysis of the plurality of patches, thereby effectively alleviating the multi-resolution dilemma of high-resolution images, where diagnostic clues are diluted in low-magnification images and computational costs are excessively high in high-magnification images.

According to the method S100, visual features are obtained from the plurality of patches in low- and high-magnification images through a visual feature extraction module, and frequency features are extracted through a frequency feature extraction module. The visual features and the frequency features are then integrated to generate a patch feature embedding.

Thereafter, in the method S100, a patch relationship information embedding may be generated by utilizing the adjacency matrix (A_{dis}) based on distance information between patches and the adjacency matrix (A_{HF}) based on the similarity of frequency features. A gated attention mechanism based on the patch relationship information embedding may then be used to select diagnostically significant top-k patches from among low-magnification patches.

According to the method S100, the features of high-magnification patches corresponding to the selected low-magnification patches may be aggregated to output a prediction value at the entire slide level. The high-resolution image analysis model trained through this method S100 may reduce computational complexity by focusing only on important areas instead of analyzing all high-magnification patches, thereby enabling diagnostic predictions with high accuracy and reliability.

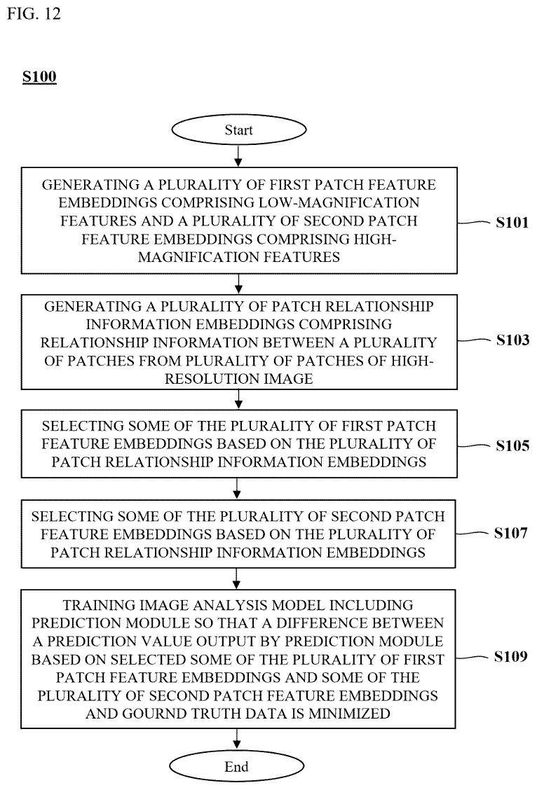

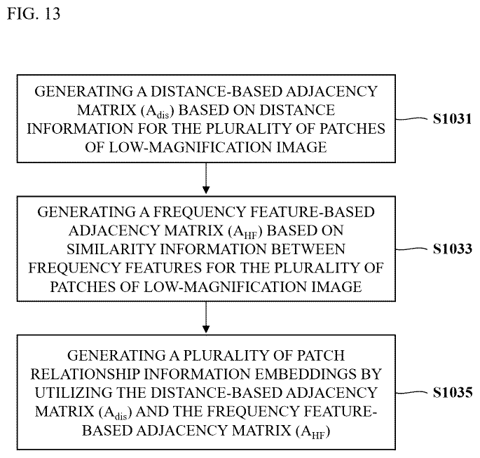

FIG. 12 is a flowchart of a method S100 for training a high-resolution image analysis model according to an embodiment. FIG. 13 is a flowchart for illustrating step S103 of FIG. 12 for generating a plurality of patch relationship information embeddings according to an embodiment.

Referring to FIG. 12, the method S100 for training the high-resolution image analysis model according to an embodiment may include: step S101 of generating a plurality of first patch feature embeddings including low-magnification features and a plurality of second patch feature embeddings including high-magnification features from the HRI; step S103 of generating a plurality of patch relationship information embeddings including relationship information between the plurality of patches from the plurality of patches of the HRI; step S105 of selecting one or more of the plurality of first patch feature embeddings based on the plurality of patch relationship information embeddings; step S107 of selecting one or more of the plurality of second patch feature embeddings based on the plurality of patch relationship information embeddings; and step S109 of training an image analysis model including a prediction module so that a difference between a prediction value output by the prediction module based on the selected one or more of the plurality of first patch feature embeddings and one or more of the plurality of second patch feature embeddings and ground truth data is minimized.

In an embodiment, the method S100 may be performed by the processor 131 included in the server computing system 130. However, an embodiment of the present disclosure is not limited thereto, and one or some steps or operations of the method S100 may be performed by the processor 111 of the user computing device 110 or the processor 151 of the training computing system 150, and the other steps or operations may be performed by the processor 131 included in the server computing system 130.

Hereinafter, for convenience of explanation, the method S100 will be described as being performed by the processor 131 included in the server computing system 130.

In step S101, the processor 131 may integrate the plurality of first visual feature embeddings (zᵢ) and the plurality of first frequency feature embeddings

$$( {z}_{i}^{h} )$$

for the plurality of patches of the low-magnification image at a first magnification for the HRI to generate the plurality of first patch feature embeddings

$$( {z}_{i}^{c} ) .$$

For instance, step S101 may include a process in which the processor 131 receives the plurality of patches of the low-magnification image from the low-magnification patching module 10, extracts visual features through the first visual feature extraction module 40, and extracts high-frequency component-based features through the first frequency feature extraction module 30.

The processor 131 may concatenate two types of embeddings, visual features and high-frequency features, obtained through the low-magnification patch feature embedding generation module 60, thereby generating the plurality of first patch feature embeddings

$$( {z}_{i}^{c} )$$

that include both general visual information of the patch and subtle morphological clues (high-frequency components) important for diagnosis.

Additionally, the processor 131 may generate the plurality of second patch feature embeddings

$$( {\overline{z}}_{ij}^{c} )$$

by integrating the plurality of second visual feature embeddings (z̅ᵢⱼ) and the plurality of second frequency feature embeddings

$$( {\overline{z}}_{ij}^{h} )$$

for the plurality of patches of the high-magnification image having a second magnification higher than a first magnification for the HRI.

This step may include a process in which the processor 131 receives the plurality of patches of the low-magnification image from the high-magnification patching module 20, extracts visual features through the second visual feature extraction module 41, and extracts high-frequency component-based features through the second frequency feature extraction module 31.

The processor 131 may concatenate the two types of embeddings, visual features and high-frequency features, obtained through the high-magnification patch feature embedding generation module 61, thereby generating the plurality of second patch feature embeddings

$$( {\overline{z}}_{ij}^{c} )$$

that include both general visual information of the patch and subtle morphological clues (high-frequency components) important for diagnosis.

In step S103, the processor 131 may generate the plurality of patch relationship information embeddings

$$( {z}_{i}^{P} )$$

that include relationship information between the plurality of patches from the plurality of patches of the HRI.

Specifically, the processor 131 may generate the plurality of patch relationship information embeddings

$$( {z}_{i}^{P} )$$

including positional information and characteristic relationship information between the plurality of patches from the plurality of patches of the HRI.

Step S103 may be performed by controlling the patch relationship information embedding generation module 50 of the processor 131 based on a plurality of pieces of patch information of the low-magnification image.

For example, referring to FIG. 13, step S103 may compute the plurality of patch relationship information embeddings

$$( {z}_{i}^{P} )$$

by utilizing the distance-based adjacency matrix (A_{dis}) based on Euclidean distance information between the plurality of patches of the low-magnification image and the frequency feature-based adjacency matrix (A_{HF}) based on the similarity of frequency feature (steps S1031, S1033, S1035).

The plurality of patch relationship information embeddings

$$( {z}_{i}^{P} )$$

encode both physical positional information and morphological similarity between the plurality of patches, and may have important properties such as invariance to slide rotation across the entire high-resolution image.

In step S105, the processor 131 may select one or more of the plurality of first patch feature embeddings

$$( {z}_{i}^{c} )$$

based on the plurality of patch relationship information embeddings

$$( {z}_{i}^{P} ) .$$

Step S105 may be performed by the processor 131 controlling the low-magnification patch feature embedding selection module 70 using the LPGA mechanism.

According to the LPGA mechanism, the processor 131 may combine the plurality of visual feature embeddings (zᵢ), the plurality of frequency feature embeddings

$$( {z}_{i}^{h} ) ,$$

and the plurality of patch relationship information embeddings

$$( {z}_{i}^{P} )$$

for the plurality of patches of the low-magnification image to generate a plurality of patch integrated embeddings

$$( {z}_{i}^{PE} ) .$$

In addition, the processor 131 may compute attention weights indicating the importance of each of the plurality of first patch feature embeddings

$$( {z}_{i}^{c} )$$

based on the plurality of patch integrated embeddings

$$( {z}_{i}^{PE} )$$

and select the top k first patch feature embeddings

$$( {z}_{i}^{c} )$$

with high weights. Through this process, the processor 131 may efficiently select low-magnification patches containing important clues for diagnosing high-resolution images.

In step S107, the processor 131 may select one or more of the plurality of second patch feature embeddings

$$( {\overline{z}}_{ij}^{c} )$$

based on the plurality of patch relationship information embeddings

$$( {z}_{i}^{P} ) .$$

Step S107 may be performed by the processor 131 controlling the high-magnification patch feature embedding selection module 71 using the LPGA mechanism.

According to the LPGA mechanism, the processor 131 may generate the plurality of patch integrated embeddings

$$( {\overline{z}}_{ij}^{PE} )$$

by combining the plurality of visual feature embeddings (z̅ᵢⱼ), the plurality of frequency feature embeddings

$$( {\overline{z}}_{ij}^{h} ) ,$$

and the plurality of patch relationship information embeddings

$$( {z}_{i}^{P} )$$

for the plurality of patches of the high-magnification image.

In addition, the processor 131 may compute attention weights indicating the importance of each of the plurality of second patch feature embeddings

$$( {\overline{z}}_{ij}^{c} )$$

based on the plurality of patch integrated embeddings

$$( {\overline{z}}_{ij}^{PE} ) ,$$

and select the top k×M² second patch feature embeddings

$$( {\overline{z}}_{ij}^{c} )$$

with high weights. Through this process, the processor 131 may efficiently select high-magnification patches containing important clues for diagnosing high-resolution images.

In this process, the LPGA arithmetic operation for the plurality of patches of the high-magnification image reuses the plurality of patch relationship information embeddings

$$( {z}_{i}^{P} )$$

obtained at low magnifications, thereby reducing the complexity of calculating the plurality of patch relationship information embeddings

$$( {z}_{i}^{P} )$$

for all of the plurality of patches of the high-magnification image. The plurality of patch relationship information embeddings

$$( {z}_{i}^{P} )$$

for the plurality of selected patches of the high-magnification image provides subtle diagnostic information for the final prediction.

In step S109, the processor 131 may train an image analysis model including the prediction module 80 to reduce or minimize a difference between the prediction value output by the prediction module 80 for the FRI based on one or some of the selected plurality of first patch feature embeddings

$$( {z}_{i}^{c} )$$

and one or some of the selected plurality of second patch feature embeddings

$$( {\overline{z}}_{ij}^{c} )$$

and the ground truth data.

Step S109 may include an integrated arithmetic operation of the MIL framework, compute a weighted sum for each of the selected low-magnification features and high-magnification features, and input these two weighted sums into a final classifier (linear head) to output a prediction value. Training may be accomplished through a process of updating the parameters of the image analysis module including the prediction module in a way that reduces or minimizes the error between the prediction value and the ground truth data. During this process, learnable parameters introduced in the LPGA arithmetic operation and learnable parameters introduced during the process of computing the plurality of patch relationship information embeddings

$$( {z}_{i}^{P} )$$

during the learning process may also be updated.

### —Method for Providing Diagnosis Responses Based on High-Resolution Image Analysis (S200)

A method S200 for providing a diagnosis response based on high-resolution image analysis may perform in-depth analysis based on the FRI and generate diagnosis responses thereto.

The method S200 for providing the diagnosis response utilizes a pre-trained high-resolution image analysis model according to the training method S100 according to an embodiment of automate diagnostic prediction for high-resolution images. For example, the high-resolution images may include tissue pathology images.

The method S200 for providing the diagnosis response may perform a step of receiving an image to be analyzed according to a user input. Subsequently, the pre-trained high-resolution image analysis model may receive the image, analyze features by patch, and synthesize these features to compute a prediction value for the entire image. This prediction value may include specific diagnosis responses, such as the presence of a disease, the grade, or the location of a lesion.

Finally, the diagnosis response output from the model may be provided to a user. Through this process, the system performing the method S200 for providing the diagnosis response may assist expert judgment or improve diagnostic efficiency, and may provide highly reliable automated diagnostic services, particularly in fields such as tissue pathology images.

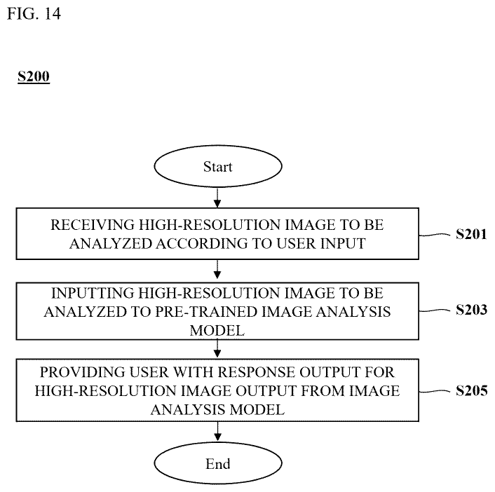

FIG. 14 is a flowchart of a method S200 for providing a diagnosis response based on high-resolution image analysis according to an embodiment.

Referring to FIG. 14, the method S200 for providing the diagnosis response based on the high-resolution image analysis according to an embodiment may include: a step S201 of receiving a high-resolution image to be analyzed according to a user input; a step S203 of inputting the high-resolution image to be analyzed to a high-resolution image analysis model pre-trained using a method for training the high-resolution image analysis model based on hierarchical patch selection of an embodiment of the present disclosure; and a step S205 of providing a user with a response output for the high-resolution image output from the high-resolution image analysis model.

In an embodiment, the method S200 may be performed by the processor 131 included in the server computing system 130. However, the present disclosure is not limited thereto, and one or some steps or operations of the method S200 may be performed by the processor 111 of the user computing device 110 or the processor 151 of the training computing system 150, while the other steps or operations of the method S200 may be performed by the processor 131 included in the server computing system 130.

Hereinafter, for convenience of explanation, the method S200 will be described as being performed by the processor 131 included in the server computing system 130.

In step S201, the processor 131 may receive the HRI to be analyzed according to a user input.

Step S201 corresponds to a process of inputting the high-resolution image provided by a user to the system 1000 to perform a diagnostic prediction.

The high-resolution image may include various types of images having high-resolution such as tissue pathology images, satellite images, aerial images, quality inspection images, surveillance camera footage, drone images, or astronomical images in the form of the WSI related to various industries such as medical industry, geographic information system (GIS), manufacturing, security, agriculture, or astronomy.

In step S203, the processor 131 may input the received high-resolution image to the pre-trained high-resolution image analysis model according to the training method S100 of an embodiment of the present disclosure.

This high-resolution image analysis model performs in-depth analysis of the image and is configured to synthesize features extracted from the plurality of patches for each of the low-magnification and high-magnification images to compute a prediction.

In step S205, the processor 131 may provide a user with a response output for the high-resolution image output from the high-resolution image analysis model.

This response output may go or extend beyond a simple classification label and include at least one of a prediction result regarding whether the high-resolution image contains a specific disease, a prediction result regarding the grade or progression of the disease, or a prediction result regarding the location of a lesion related to the disease.

In step S205, the processor 131 may provide an image highlighting a portion of the high-resolution image utilized to decide the response output, along with the response output, to enhance the reliability of the diagnosis response and the interpretability of the model.

These highlighted areas visually represent the portions utilized as important basis for the final diagnostic determination during a process of the image analysis model synthesizing features extracted from the plurality of patches of each low- and high-magnification image to derive a prediction value.

For example, some highlighted areas of the high-resolution image may correspond to some of the low-magnification features and some of the high-magnification features of the high-resolution image that have been utilized in the arithmetic operation to decide the diagnosis response, which is the response output for the high-resolution image.

Some highlighted areas of the high-resolution image may correspond to some of the plurality of patch feature embeddings selected by the processor 131 using the low-magnification patch feature embedding selection module 70 and the high-magnification patch feature embedding selection module 71.

For example, when the high-resolution image includes a tissue pathology image, highlighting and showing a specific patch area densely populated with cancer cells allow a user to clearly understand what visual information the response output of the image analysis model is based on. This function may help users trust the determination of the model more and help experts efficiently review a diagnostic process.

### —Method for Training Time-Series Analysis Model for Plurality of High-Resolution Images (S300)

According to an embodiment, a method S300 for training a time-series analysis model for the plurality of high-resolution images performs time-series data analysis on deep analysis prediction values for each of the plurality of HRIs, thereby providing a method for training a model capable of providing reliable predictive diagnosis responses for the plurality of high-resolution images.

The time-series analysis model trained according to method S300 may comprehensively grasp or detect image change patterns across a plurality of points in time, going beyond individual analysis of high-resolution images.

According to the method S300, the time-series analysis model performs efficient analysis based on hierarchical patch selection for each high-resolution image and configures the resulting analysis prediction values into time-series data aligned in chronological order. This configured time-series data may be input to the time-series analysis model, such as a Transformer, to learn change patterns and long-term dependencies over time in images. The time-series analysis model trained through this process may provide precise predictive diagnosis responses that reflect temporal changes, such as change trends, beyond simple single-point-level predictions.

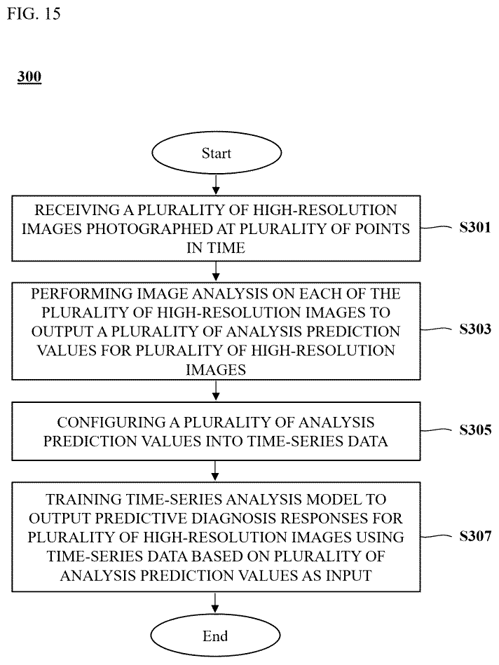

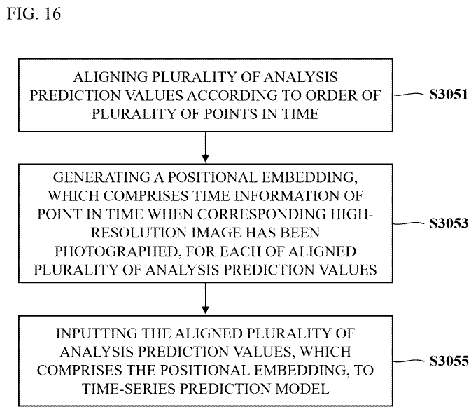

FIG. 15 is a flowchart of a method S300 for training a time-series analysis model for a plurality of high-resolution images according to an embodiment. FIG. 16 is a flowchart for illustrating step S303 of FIG. 15 of configuring a plurality of analysis prediction values into time-series data according to an embodiment.

In an embodiment, the method S300 may be performed by the processor 131 included in the server computing system 130. However, the present disclosure is not limited thereto, and one or more steps or operations of the method S300 may be performed by the processor 111 of the user computing device 110 or the processor 151 of the training computing system 150, while the other steps or operations of the method S300 may be performed by the processor 131 included in the server computing system 130.

Hereinafter, for convenience of explanation, the method S300 is described as being performed by the processor 131 included in the server computing system 130.

Referring to FIG. 15, the method S300 may include: a step S301 of receiving a plurality of high-resolution images (HRIs) photographed at a plurality of points in time; a step S303 of performing image analysis based on hierarchical patch selection on each of the plurality of HRIs to output a plurality of analysis prediction values for the plurality of HRIs; a step S305 of configuring the plurality of analysis prediction values into time-series data; and a step S307 of training the time-series analysis model to output predictive diagnosis responses for the plurality of HRIs using the time-series data based on the plurality of analysis prediction values as an input.

In step S301, the processor 131 may receive the plurality of HRIs photographed at the plurality of points in time for training.

Step S301 corresponds to a process of inputting a data set with a temporal order, such as the plurality of HRIs photographed at different points in time, to the system. For example, the processor 131 may receive a plurality of tissue pathology images photographed at a plurality of points in time of a patient.

In step S303, the processor 131 may perform image analysis based on hierarchical patch selection on each of the received plurality of HRIs and output a plurality of analysis prediction values for each of the plurality of HRIs.

In step S303, the processor 131 may analyze each of the plurality of HRIs using the hierarchical patch selection-based image analysis module 401. By step S303, the processor 131 may efficiently select diagnostically significant patches from low-magnification images of each of the plurality of HRIs and synthesize the features of the corresponding high-magnification patches to compute a prediction value for each image.

In step S305, the processor 131 may configure the plurality of analysis prediction values output for each of the plurality of HRIs into time-series data.

Step S305 corresponds to a process of refining data so that the time-series analysis model may learn temporal information.

In detail, referring to FIG. 16, in step S305, the plurality of analysis prediction values are aligned according to the order of photographed time (S3051). For each prediction value, a positional embedding containing time information of a point in time when the corresponding high-resolution image has been photographed is generated (S3053). The aligned plurality of analysis prediction values including the positional embedding may be provided as input data for the time-series prediction model (S3055).

In step S307, the processor 131 may train the time-series analysis model using the time-series data based on the plurality of analysis prediction values as an input. This time-series analysis model may be trained to predict a diagnosis response at the next point in time by learning the temporal patterns and long-term dependencies of the time-series data.

For example, the processor 131 may train an attention-based model, such as a Transformer, to predict the diagnosis response that reflects temporal changes, such as the pathological change trends and the likelihood of recurrence of a patient. Finally, this trained time-series analysis model may output the predictive diagnosis responses for the plurality of HRIs to be analyzed newly input to the plurality of HRIs.

—Method for Providing Diagnosis Response based on Time-Series Analysis for Plurality of High-Resolution Images (S400)

According to an embodiment, a method S400 for providing a diagnosis response based on time-series analysis for the plurality of high-resolution images performs time-series data analysis on in-depth analysis prediction values for each of the plurality of HRIs, thereby providing reliable predictive diagnosis responses for the plurality of HRIs.

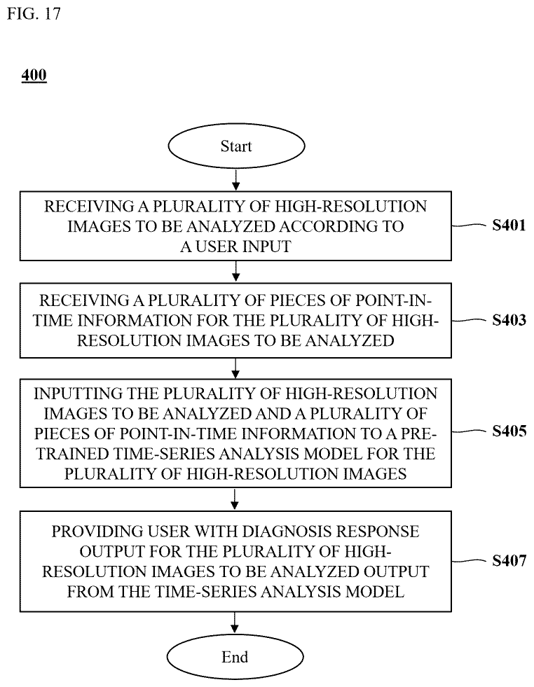

FIG. 17 is a flowchart of a method S400 for providing a diagnosis response based on time-series analysis for a plurality of high-resolution images according to an embodiment.

In an embodiment, the method S400 may be performed by the processor 131 included in the server computing system 130. However, the present disclosure is not limited thereto, and one or more steps or operations of the method S400 may be performed by the processor 111 of the user computing device 110 or the processor 151 of the training computing system 150, and the other steps or operations may be performed by the processor 131 included in the server computing system 130.

Hereinafter, for convenience of explanation, the method S400 will be described as being performed by the processor 131 included in the server computing system 130.

Referring to FIG. 17, the method S400 may include: a step S401 of receiving a plurality of high-resolution images (HRIs) to be analyzed according to a user input; a step of S403 of receiving a plurality of pieces of point-in-time information for the plurality of HRIs to be analyzed; a step of S405 of inputting the plurality of HRIs to be analyzed and the plurality of pieces of point-in-time information to a time-series analysis model for the plurality of HRIs trained by the method S300 of FIG. 15; and a step of S407 of providing a user with a diagnosis response output for the plurality of HRIs to be analyzed output from the time-series analysis model.

In step S401, the processor 131 may receive the plurality of HRIs to be analyzed according to the user input. Step S401 corresponds to a process of inputting high-resolution images with a temporal order, such as tissue pathology images photographed over a plurality of different points in time or satellite images recording changes in an urban area, to the system 1000.

In step S403, the processor 131 may receive the plurality of pieces of point-in-time information for the plurality of high-resolution images to be analyzed. The point-in-time information indicates the precise temporal order in which each image was acquired and is essential metadata for time-series data analysis. This information may be utilized to generate positional embeddings indicating the temporal positions of the images at the time of configuring time-series data.

In step S405, the processor 131 may input the plurality of HRIs to be analyzed and the plurality of pieces of point-in-time information to a pre-trained time-series analysis model. This time-series analysis model may be trained by the hierarchical patch selection-based image analysis module 401 to efficiently analyze each FRI and compute analysis prediction values for each point-in-time. These analysis prediction values may then be input to the time-series analysis model after being configured as time-series data, along with a plurality of positional embeddings containing temporal order information at the points in time when the plurality of high-resolution images were photographed.

In step S407, the processor 131 may provide a user with a diagnosis response output from the time-series analysis model. This response may include precise and reliable information that comprehensively reflects images from a plurality of points in time, such as disease progression over time, the likelihood of disease recurrence, or future change trends, beyond a simple prediction value at a single point in time. Through this process, the system 1000 may assist expert judgment and provide long-term predictions, thereby enhancing diagnostic efficiency and accuracy.

The embodiments of the present disclosure described above may be implemented in the form of program commands which may be executed through various types of computer constituting elements and recorded in a computer-readable recording medium. The computer-readable recording medium may include program commands, data files, and data structures separately or in combination thereof. The program commands recorded in the computer-readable recording medium may be those designed and configured specifically for the present disclosure or may be those commonly available for those skilled in the field of computer software. Examples of a computer-readable recoding medium may include magnetic media such as hard-disks, floppy disks, and magnetic tapes; optical media such as CD-ROMs and DVDs; and hardware devices specially designed to store and execute program commands such as ROM, RAM, and flash memory. Examples of program commands include not only machine codes such as those generated by a compiler but also high-level language codes which may be executed by a computer through an interpreter and the like. The hardware device may be replaced with by one or more software modules to perform the operations of the present disclosure, and vice versa.

Specific executions described in the present disclosure are exemplary embodiments and the scope of the present disclosure is not limited even by any method. For brevity of the specification, descriptions of conventional electronic configurations, control systems, software, and other functional aspects of the systems may be omitted. Further, connection or connection members of lines among components exemplarily represent functions connections and/or physical or circuitry connections and may be represented as various functional connections, physical connections, or circuitry connections which are replaceable or added in an actual device. Further, unless otherwise specified, such as “essential” or “important,” the connections may not be components particularly required for application of the present disclosure.

Further, in the detailed description of the present disclosure, which is described, while the present disclosure has been described with respect to the preferred embodiments, it will be understood by those skilled in the art or those skilled in the art having ordinary knowledge in the technical field that various changes and modifications of the present disclosure may be made without departing from the spirit and the technical scope of the present disclosure described in the following claims. Accordingly, the technical scope of the present disclosure should not be limited to the contents described in the detailed description of the present disclosure but should be defined by the claims.

Various embodiments of the present disclosure may enhance the performance-to-cost ratio and reliability across various industrial fields requiring high-resolution image analysis, such as medical and manufacturing industries, by capturing important clues through frequency analysis of a low-magnification image of an analysis target image and efficiently selecting key patches of a high-magnification image of the same analysis target image based thereon and using the same to produce a response output, thereby increasing or maximizing computational efficiency while maintaining accuracy. Therefore, various embodiments of the present disclosure have industrial applicability.

What is claimed is:

1. A computer-implemented method comprising:
receiving an activation signal for accessing data stored in memory;
in response to the receiving of the activation signal, accessing a diagnostic image stored in the memory;
loading the diagnostic image from the memory;
generating at least one response to the diagnostic image using at least one artificial intelligence model configured to receive the diagnostic image as an input, the at least one artificial intelligence model being pre-trained to perform image analysis based on data that integrates one or more of low-magnification features and one or more of high-magnification features of the diagnostic image; and
inputting the at least one response to the diagnostic image to at least one subsequent processing component.

2. The computer-implemented method of claim 1, further comprising manifesting, by the at least one subsequent processing component, the at least one response to the diagnostic image through at least one user interface.

3. The computer-implemented method of claim 1, further comprising providing, through the user interface, an image in which a partial area of the diagnostic image is highlighted, wherein the partial area of the diagnostic image corresponds to one or more of the low-magnification features of the diagnostic image and one or more of the high-magnification features of the diagnostic image utilized in an arithmetic operation for determining the response to the diagnostic image.

4. The computer-implemented method of claim 1, wherein the response to the diagnostic image has one or both of interactive text or voice.

5. The computer-implemented method of claim 1, further comprising:
receiving a plurality of diagnostic images according to a user input;
receiving a plurality of pieces of point-in-time information for the plurality of diagnostic images;
inputting the plurality of diagnostic images and the plurality of pieces of point-in-time information to the at least one pre-trained artificial intelligence model; and
providing a user with a response output for the plurality of diagnostic images output from the at least one pre-trained artificial intelligence model.

6. The computer-implemented method of claim 5, wherein:
each of the plurality of diagnostic images comprises a tissue pathology image for the user, and
the response output comprises at least one of a progression of a disease of the user, a grade of the disease, a likelihood of recurrence of the disease, or a future change trend of the disease.

7. The computer-implemented method of claim 5, wherein the at least one artificial intelligence model is pre-trained for the plurality of diagnostic images by operations comprising:
outputting a plurality of analysis prediction values for the plurality of diagnostic images by performing the image analysis on the plurality of diagnostic images photographed at a plurality of points in time using the at least one artificial intelligence model;
configuring the plurality of analysis prediction values into time-series data; and
training the at least one artificial intelligence model for the plurality of diagnostic images to output predictive diagnosis responses for the plurality of diagnostic images using the time-series data based on the plurality of analysis prediction values as input.

8. The computer-implemented method of claim 7, wherein the configuring of the plurality of analysis prediction values into the time-series data comprises:
aligning the plurality of analysis prediction values according to an order of the plurality of points in time;
generating a positional embedding, which comprises time information of a point in time when each of the plurality of diagnostic images was photographed, for each of the plurality of aligned analysis prediction values; and
inputting the plurality of aligned analysis prediction values, which comprises the positional embedding, to the at least one artificial intelligence model for the plurality of diagnostic images.

9. The computer-implemented method of claim 1, wherein the at least one artificial intelligence model is pre-trained by operations comprising:
generating a plurality of first patch feature embeddings comprising low-magnification features and a plurality of second patch feature embeddings comprising high-magnification features from the diagnostic image;
generating a plurality of patch relationship information embeddings comprising relationship information between a plurality of patches of the diagnostic image;
selecting one or more of the plurality of first patch feature embeddings based on the plurality of patch relationship information embeddings;
selecting one or more of the plurality of second patch feature embeddings based on the plurality of patch relationship information embeddings; and
training the at least one artificial intelligence model to reduce a difference between a prediction value output based on the selected one or more of the plurality of first patch feature embeddings and the selected one or more of the plurality of second patch feature embeddings and ground truth data.

10. The computer-implemented method of claim 9, wherein the generating of the plurality of patch relationship information embeddings comprises:
generating a distance-based adjacency matrix based on distance information between the plurality of patches of a low-magnification image for the diagnostic image;
generating a frequency feature-based adjacency matrix based on similarity information between frequency features for the plurality of patches of the low-magnification image for the diagnostic image; and
generating the plurality of patch relationship information embeddings comprising positional information and characteristic relationship information between the plurality of patches by utilizing the distance-based adjacency matrix and the frequency feature-based adjacency matrix.

11. The computer-implemented method of claim 10, wherein the generating of the frequency feature-based adjacency matrix comprises:
computing similarity between a plurality of frequency feature embeddings for the plurality of patches of the low-magnification image; and
generating the frequency feature-based adjacency matrix based on the similarity between the plurality of frequency feature embeddings and the distance information between the plurality of patches of the low-magnification image.

12. The computer-implemented method of claim 10, wherein the generating of the plurality of patch relationship information embeddings comprises:
acquiring a plurality of positional feature vectors encoding geometric positional information between the plurality of patches of the low-magnification image by performing spectral analysis on the distance-based adjacency matrix; and
generating the plurality of patch relationship information embeddings by performing a neural network arithmetic operation that learns a graph structure on one or more of the plurality of acquired positional feature vectors selected according to a predetermined condition and the frequency feature-based adjacency matrix.

13. The computer-implemented method of claim 12, wherein the acquiring of the plurality of positional feature vectors comprises computing a plurality of eigenvectors for a graph Laplacian of the distance-based adjacency matrix as the plurality of positional feature vectors using an equation:
$$Δ = {I}_{n} - {D}_{dis}^{- 1 / 2} {A}_{dis} {D}_{dis}^{- 1 / 2} = {U}^{T} ΛU ,$$
wherein Δ is the graph Laplacian, Iₙ is an identity matrix, D_{dis} is a degree matrix, A_{dis} is the distance-based adjacency matrix, U is an eigenvector matrix, and Λ is an eigenvalue matrix.

14. The computer-implemented method of claim 9, wherein the generating of the plurality of first patch feature embeddings and the plurality of second patch feature embeddings comprises:
generating the plurality of first patch feature embeddings by integrating a plurality of first visual feature embeddings and a plurality of first frequency feature embeddings for a low-magnification image of the diagnostic image; and
generating the plurality of second patch feature embeddings by integrating a plurality of second visual feature embeddings and a plurality of second frequency feature embeddings for a high-magnification image of the diagnostic image.

15. The computer-implemented method of claim 14, wherein the selecting of one or more of the plurality of first patch feature embeddings comprises:
computing a plurality of weights for the plurality of patches of the low-magnification image based on a plurality of patch integrated embeddings that integrate the plurality of first visual feature embeddings, the plurality of first frequency feature embeddings, and the plurality of patch relationship information embeddings; and
selecting k first patch feature embeddings corresponding to top k weights (k is a positive integer) among the plurality of weights from the plurality of first patch feature embeddings.

16. The computer-implemented method of claim 15, wherein the selecting of one or more of the plurality of second patch feature embeddings comprises:
computing a plurality of weights for the plurality of patches of the high-magnification image based on a plurality of patch integrated embeddings that integrate the plurality of second visual feature embeddings, the plurality of second frequency feature embeddings, and the plurality of patch relationship information embeddings; and
selecting k×M² second patch feature embeddings corresponding to top k×M² weights (M is a relative magnification of a high magnification to a low magnification) among the plurality of weights from the plurality of second patch feature embeddings.

17. The computer-implemented method of claim 16, wherein the training of the at least one artificial intelligence model comprises:
computing a first weighted sum of the selected k first patch feature embeddings based on the top k weights for the plurality of patches of the low-magnification image;
computing a second weighted sum of the selected k×M² second patch feature embeddings based on the top k×M² weights for the plurality of patches of the high-magnification image; and
training the at least one artificial intelligence model to reduce the difference between the prediction value output using a result of a predetermined arithmetic operation applied to the first weighted sum and the second weighted sum and the ground truth data.

18. A system comprising:
memory configured to store instructions that are executable; and
at least one processor configured to execute configured to execute one or more of the instructions to perform operations comprising:
receiving an activation signal for accessing data stored in the memory;
in response to the receiving of the activation signal, accessing a diagnostic image stored in the memory;
loading the diagnostic image from the memory;
generating at least one response to the diagnostic image using at least one artificial intelligence model configured to receive the diagnostic image as an input, the at least one artificial intelligence model being pre-trained to perform image analysis based on data that integrates one or more of low-magnification features and one or more of high-magnification features of the diagnostic image; and
inputting the at least one response to the diagnostic image to at least one subsequent processing component.

19. The system of claim 18, further comprising:
a plurality of neurons configured as an array comprising at least one register, at least one programmable logic, and at least one input interface;
a plurality of synaptic circuits storing synaptic weights that adjust concatenation strengths between the plurality of neurons; and
at least one routing network configured to control data flow between the plurality of neurons,
wherein each of the plurality of neurons is concatenated to at least another neuron of the plurality of neurons through the routing network and comprises a field programmable gate array (FPGA) implementation for a predetermined artificial neural network configured to provide a transfer path for the weights.

20. The system of claim 18, further comprising:
a plurality of neurons organized as an array comprising at least one register, at least one microprocessor, and at least one input; and
a plurality of synaptic circuits configured to store synaptic weights that adjust concatenation strengths between the plurality of neurons,
wherein each of the plurality of neurons comprises an application specific integrated circuit (ASIC) for a predetermined artificial neural network concatenated to at least another neuron of the plurality of neurons through one of the plurality of synaptic circuits.
